(19) United States
(12) Patent Application Publication
(10) Pub. No.: US 2024/0308969 A1
(43) Pub. Date: Sep. 19, 2024

(54) INVERSE ELECTRON DEMAND DIELS-ALDER TYPE HETEROCYCLE SYNTHESIS WITH 1,2,3-TRIAZINE 1-OXIDES

(71) Applicant: Board of Regents, The University of Texas System, Austin, TX (US)

(72) Inventors: Michael P. Doyle, San Antonio, TX (US); Luca De Angelis, San Antonio, TX (US); Soumen Biswas, San Antonio, TX (US); Gildardo Rivera, Reynose (MX)

(21) Appl. No.: 18/438,460

(22) Filed: Feb. 10, 2024

Related U.S. Application Data
(60) Provisional application No. 63/444,662, filed on Feb. 10, 2023.

(51) Int. Cl. C07D 239/28; C07D 211/90; C07D 401/04; C07D 403/04; C07D 487/04; C07J 43/00

(52) U.S. Cl. CPC C07D 239/28; C07D 211/90; C07D 401/04; C07D 403/04; C07D 487/04; C07J 43/003

(57) ABSTRACT

Embodiments are directed to practical strategies for inverse electron demand Diels-Alder (IEDDA) reactions with 1,2,3-triazine 1-oxides. These bench stable N-oxides undergo unprecedented nucleophilic addition with dienophile to form polysubstituted heteroarenes in high yield. Rapid at room temperature, these catalyst free reactions offer a diversity of structural modifications for heterocyclic syntheses.

### RELATED APPLICATIONS

This Application is a non-provisional filing of and claims priority to U.S. Provisional patent application No. 63/444,662 filed Feb. 10, 2023, which is incorporated herein by reference in its entirety.

### STATEMENT REGARDING FEDERALLY FUNDED RESEARCH

None.

### FIELD OF THE INVENTION

Embodiments are directed generally to chemical synthesis and in particular inverse electron demand Diels-Alder reactions.

### BACKGROUND

The inverse electron demand Diels-Alder (IEDDA) reaction with 1,2,3-triazines has significantly broadened structural diversity for the synthesis of pyrimidine and pyridine heterocycles (Zhang et al., Chem. Rev. 2021, 121, 14555-93; Foster and Willis, Chem. Soc. Revs. 2013, 42, 63-76). This transformation readily occurs by a concerted or stepwise cycloaddition coupled with the loss of dinitrogen (Yang et al., J. Am. Chem. Soc. 2017, 139, 18213-21; Wu et al., J. Am. Chem. Soc. 2022, 144, 10921-28), and the product formed retains the structural connectivity at the triazine 4-, 5-, and 6-positions (FIG. 1)(Anderson and Boger, J. Am. Chem. Soc. 2011, 133, 13385-2292; Anderson et al., Org. Lett. 2014, 16, 5084-87; Glinkerman and Boger, Org. Lett. 2015, 17, 4002-05; Zhang et al., J. Org. Chem. 2019, 84, 9397-445). Consequently, access to relevant pyrimidine and pyridine heterocycles is determined by the structural design of the reactant 1,2,3-triazine, and this design is dependent on the synthetic protocol for the 1,2,3-triazine that is employed. Several methods have become available for the synthesis of 1,2,3-triazines (Zhang et al., Chem. Rev. 2021, 121, 14555-93; Foster and Willis, Chem. Soc. Revs. 2013, 42, 63-76; Döpp and Döpp, 1,2,3-Triazines and their Benzo Derivatives. In Comprehensive Heterocyclic Chemistry III; Katritzky, Ramsden, Scriven, Taylor, Eds .; Elsevier: Oxford, 2008), with the one from 1,2,3-triazine 1-oxides by deoxygenation recently published (Rivera et al., Org. Lett. 2022, 24, 6543-47), but relatively harsh oxidative ring-expansion of 1-aminopyrazoles (Ohsawa et al., J. Org. Chem. 1985, 50, 5520-23) and intramolecular cyclization of azidoalkenoates (Sugimura et al., Org. Lett. 2018, 20, 3434-3437) have been the dominant strategies. Each methodology has its advantages, dependent on access to relevant reactants, but also inherent synthetic disadvantages.

There remains a need for additional methods for producing heterocyclic molecules.

### SUMMARY

Embodiments described herein are directed to strategies for inverse electron demand Diels-Alder (IEDDA) reactions with 1,2,3-triazine 1-oxides. These bench stable N-oxides undergo unprecedented nucleophilic addition with dienophile to form polysubstituted heteroarenes in high yield. Rapid at room temperature, these catalyst free reactions offer a diversity of structural modifications for heterocyclic syntheses.

Certain embodiments are directed to an improved chemical process. The chemical process including, but not limited to (a) combining a first 1,2,3-triazine 1-oxide reactant with a compatible second reactant in a solvent forming a reaction solution; (b) incubating the reaction solution under conditions to form a product and nitrous oxide; and (c) isolating the product. In certain aspects the 1,2,3-triazine 1-oxide has the general structure of Formula I:

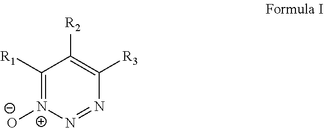

wherein R1, R2, and R3 are independently hydrogen, substituted or unsubstituted cycloalkyl, substituted or unsubstituted heterocyclyl, substituted or unsubstituted monocyclic aryl, substituted or unsubstituted polycyclic aryl, ester, or aldehyde. In certain aspects R1 is hydrogen, R2 is aryl, polycyclic aryl, cyclopropyl, or functionalized alkyl without a H at the alpha position and R3 is an ester. In certain aspects the ester can be converted to other groups (e.g., CH₂OH, COOH, CONH₂, aldehyde, or ketone) or prepared as a sulfone or phosphonate. In certain aspects the solvent is dioxane, tetrahydrofuran, methyl chloride, acetonitrile, or dichloromethane. The the reaction solution can be incubated at a temperature of 10 to 35° C. In certain aspects the reaction solution is incubated at a temperature of 20 to 25° C. The product is a nitrogen containing monocyclic heterocycle or polycyclic heterocycle. In certain aspects the product is isolated by chromatography, for example silica gel chromatography. The reaction can substantially complete within 10 to 120 minutes. In certain aspects the reaction is substantially complete within 20 to 40 minutes.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of synthetic methods for the preparation and applications of 1,2,3-triazenes.

FIG. 2. Illustrates a practical strategy for inverse electron demand Diels-Alder (IEDDA) reactions with 1,2,3-triazine 1-oxides is described. These bench stable N-oxides undergo unprecedented nucleophilic addition with dienophile to form polysubstituted heteroarenes in high yield. Rapid at room temperature, these catalyst free reactions offer a diversity of structural modifications for heterocyclic syntheses.

FIG. 3. Illustration of examples of 1,2,3-triazine 1-oxides.

FIG. 4. Illustration of ORTEP drawing of 3a, 4a, 11i showing thermal ellipsoids at the 50% probability level.

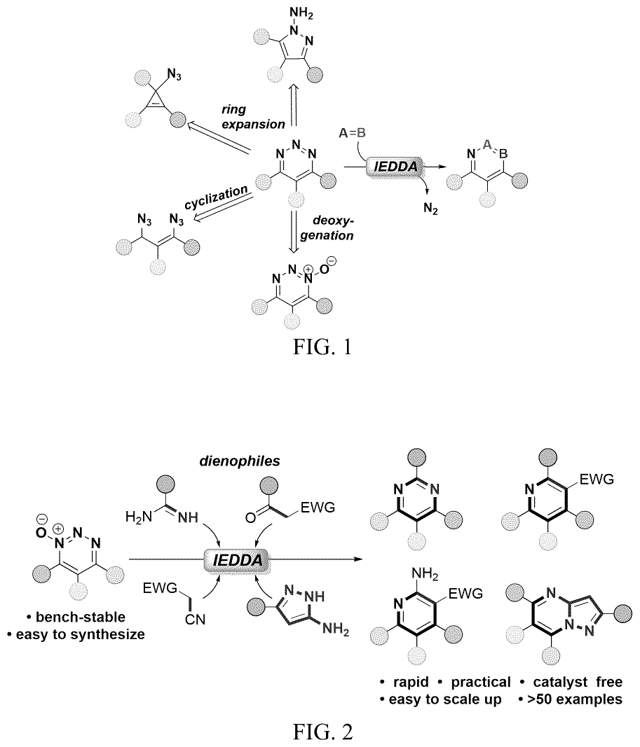

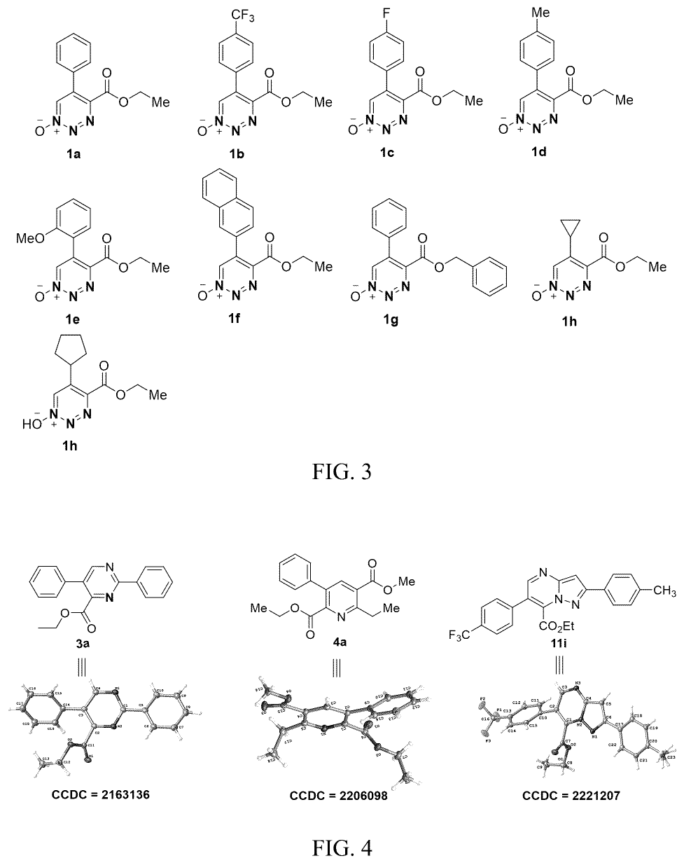

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

The inventors have recently reported a mild and convenient methodology for the synthesis of 1,2,3-triazine 1-oxides from vinyldiazoacetates and tert-butyl nitrite (De Angelis et al., Org. Lett. 2021, 23, 6542-46). This transformation places a carboxylate group at the 4-position and is general for alkyl or aryl substituents at the 5- and 6-positions. It was not known if the IEDDA reaction could be as general in scope with 1,2,3-triazine 1-oxides as with 1,2,3-triazines. The formal difference between these two reactions is that, whereas triazines expel dinitrogen, the triazine 1-oxides release nitrous oxide (Eq. 1). Described herein is the broad scope of 1,2,3-triazine 1-oxides in the formation heterocyclic compounds by what is known as the inverse electron demand Diels-Alder (IEDDA) reaction with a general group of A=B reactants that have been successful with 1,2,3-triazines, and the outcomes of IEDDA reactions of 1,2,3-triazine 1-oxides are compared with identically constituted 1,2,3-triazines.

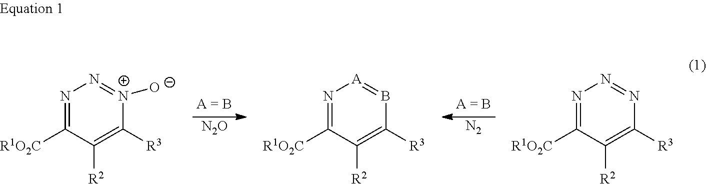

1,2,3-Triazine 1-oxides are remarkably effective substrates for inverse electron demand Diels-Alder reactions. Formed from vinyldiazoacetates via reaction with tert-butyl nitrite, these stable heterocyclic compounds undergo clean nucleophilic addition with amidines to form pyrimidines, with β-ketocarbonyl compounds and related nitrile derivatives to form polysubstituted pyridines, and with 2-aminopyrroles to form pyrazolo[1,5-a]pyrimidines, in high yield. These practical reactions are rapid at room temperature, base catalyzed, and offer a diversity of structural modifications.

4-ethylcarboxylato-5-phenyl-1,2,3-triazine 1-oxide (1a) was selected as the model substrate for study. The phenyl group at the 5-position enhances reactivity in IEDDA reactions of 1,2,3-triazines relative to hydrogen, but is not as activating as the often-used —COOR group (Anderson and Boger, J. Am. Chem. Soc. 2011, 133, 13385-12292). Because we have access to the corresponding 1,2,3-triazine 2a by phosphite-induced deoxygenation of 1a (Rivera et al., Org. Lett. 2022, 24, 6543-47), IEDDA reactions of the 1,2,3-triazine-1-oxide can be directly compared with reactions of the corresponding 1,2,3-triazine. A classic IEDDA reaction with 1,2,3-triazines is that with benzamidine to give the polysubstituted pyrimidine corresponding to the replacement of dinitrogen on the 1,2,3-triazine by the imido group from the benzamidine (Anderson and Boger, J. Am. Chem. Soc. 2011, 133, 13385-13392; Anderson et al., Org. Lett. 2014, 16, 5084-87; Glinkerman and Boger, Org. Lett. 2015, 17, 4002-05; Anderson and Boger, Org. Lett. 2011, 13, 2492-94; Glinkerman and Boger, Org. Lett. 2018, 20, 2628-31; Quiñones et al., J. Org. Chem. 2021, 86, 13465-74), whose value has been demonstrated in numerous synthetic applications (Duerfeldt and Boger, J. Am. Chem. Soc. 2014, 136, 2119-25; Lee et al., Tetrahedron 2015, 71, 5897-5905; Glinkerman and Boger, J. Am. Chem. Soc. 2016, 138, 12408-13). Treatment of either 1a or 2a with benzamidine (1.2 equiv.) in dioxane at room temperature under the same conditions formed pyrimidine 3a as the sole product, and both in very high yield (Scheme 1). Product formation was about 50% faster with 1a than with 2a. Dioxane was used instead of acetonitrile as the reaction solvent due to a better reaction time and yield (see Table 2); and the 1,2,3-triazine 1-oxide, rather than benzamidine, was the limiting reagent. It is established that reactions of triazines with amidines occur rapidly at room temperature (Quiñones et al., J. Org. Chem. 2021, 86, 13465-74), as is their high conversions to a diverse array of pyrimidine products, but the use of 1a as a representative of a new array of triazines and their precursor 1-oxides offers new advantages for IEDDA and related reactions.

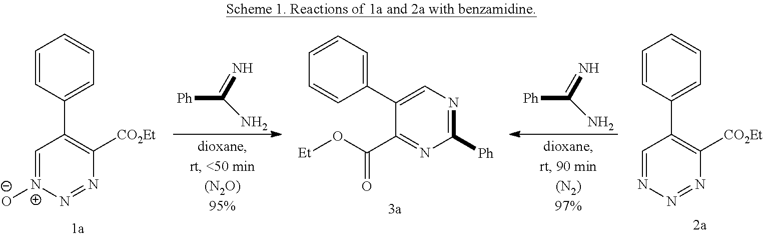

The potential scope for pyrimidine formation from diverse 1,2,3-triazine 1-oxides was examined, and the outcome is given in Scheme 2. These reactions occur with relatively rapid rates at room temperature. Pyrimidine product yields with 5-substituted triazine 1-oxides were high, although somewhat lower with a methyl substituent because, due to consideration of solubility, this methyl amidine was used as its hydrochloride salt with an added equivalent amount of base.

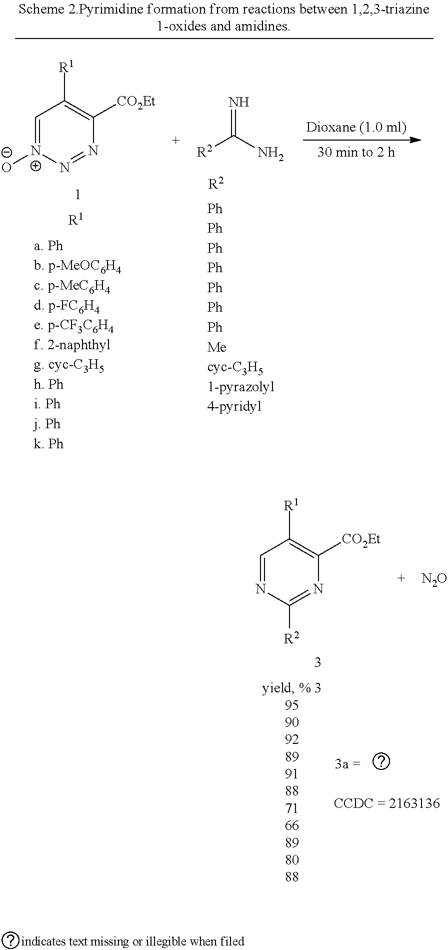

To ascertain the generality of the IEDDA-type reactions with nucleophilic “dienophiles” we undertook a survey of representative IEDDA-compatible substrates, most of which have been reported in at least one example to undergo pyridine formation with 1,2,3-triazines, although at higher temperatures (Zhang et al., Chi. Chem. Lett. 2021, 32, 393-96). In each case both the 1,2,3-triazine-1-oxide 1a and its corresponding 1,2,3-triazine 2a formed the same compound when reaction with the same nucleophile was performed under the same conditions. Also, like the outcome of their reactions with benzamidines (Table 1), the initial nucleophilic addition to both 1a and 2a occurs at the 6-position, rather than at the 4-position. When treated with 1.2 equiv. of methyl 3-oxopentanoate at room temperature in the presence of DBU as the base, 1a undergoes displacement of N₂O, which was identified by infrared spectroscopic analysis of the gaseous effluent from the reaction. This reaction forms the polysubstituted pyridine 5a as the sole product in very high yield (Scheme 3). The same transformation occurs with 1,2,3-triazine 2a, although at a much slower rate, to also form 5a in comparable high yield. Similar reactions with 2,4-pentanedione produced the corresponding pyridines in variable yields, but with the rate of reaction faster with the triazine 1-oxide than with the triazine. The previously unreported reactions with 1,3-cyclohexadione that forms the fused ring pyridine 7a showed similar characteristics with a higher product yield and a shorter reaction time with 1a than with 2a; however, 1,3-cyclopentadione and 1,3-indandione does not undergo a similar IEDDA reaction under these same conditions. Nitrile-activated systems also give pyridine products with both 1a and 2a, but in these cases their reaction times are identical or nearly identical, and their product yields are similar.

TABLE 1
Comparison of 1,2,3-triazine 1-oxide 1a with 1,2,3-triazine 2a in IEDDA reactions with
nucleophiles.

| | | | time, % yield^{b} | time, % yield^{b} |
|---|---|---|---|---|
| A═B reactant | conditions | product | from 1a | from 2a |
| 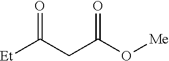 | DBU (1.0 eq.), DCM (1.0 mL), rt | 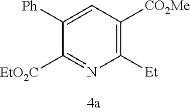 [SMILES: CCOC(=O)c1nc(CC)c(C(=O)OC)cc1-c1ccccc1] | 25 min, 94% | 1.5 h, 92% |
| 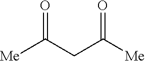 | DBU (1.0 eq.), DCM (1.0 mL), rt | 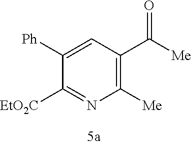 [SMILES: CCOC(=O)c1nc(C)c(C(C)=O)cc1-c1ccccc1] | 3 h, 62% | 6 h, 82% |
| 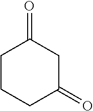 | DBU (1.0 eq.), DCM (1.0 mL), rt | 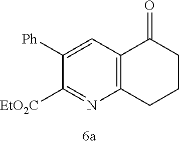 [SMILES: CCOC(=O)c1nc2c(cc1-c1ccccc1)C(=O)CCC2] | 2.5 h, 80% | 18 h, 58% |
| 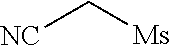 | DBU (1.0 eq.), DCM (1.0 mL), rt | 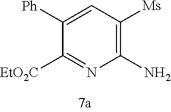 [SMILES: CCOC(=O)c1nc(N)c(S(C)(=O)=O)cc1-c1ccccc1] | 1.5 h, 89% | 1.5 h, 90% |
| 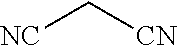 | DBU (1.0 eq.), DCM (1.0 mL), rt | 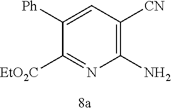 [SMILES: CCOC(=O)c1nc(N)c(C#N)cc1-c1ccccc1] | 15 min, 99% | 15 min, 98% |
| 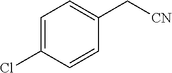 | DBU (1.0 eq.), DCM (1.0 mL), rt | 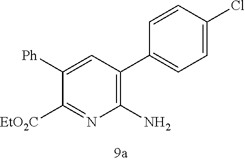 [SMILES: CCOC(=O)c1nc(N)c(-c2ccc(Cl)cc2)cc1-c1ccccc1] | 24 h, 50% | 24 h, 42% |
| 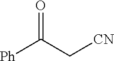 [SMILES: N#CCC(=O)c1ccccc1] | DBU (1.0 eq.), DCM (1.0 mL), rt | 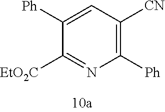 [SMILES: CCOC(=O)c1nc(-c2ccccc2)c(C#N)cc1-c1ccccc1] | 24 h, 61% | 24 h, 64% |
| 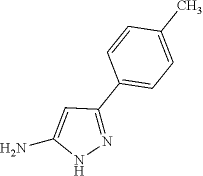 | Et₃N (2.0 eq.), DCM (1.0 ml), rt | 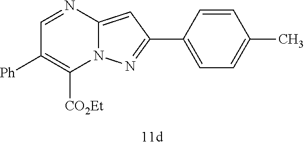 [SMILES: CCOC(=O)c1c(-c2ccccc2)cnc2cc(-c3ccc(C)cc3)nn12] | 72 h, 70% | 72 h, trace |

^{a}Reactions were performed on a 0.1 mmmol scale with 1a or 2a as the limiting reagent. A 50% molar excess of the nucleophile was employed.
^{b}Yields are isolated yields; time is lenght of time until reactant 1a or 2a was no longer visible by TLC.

5-Aminopyrazoles, which are known to undergo IEDDA reactions with 1,3,5-triazines to form pyrazolo[4,3-d]pyrimidines (Xu et al., Acc. Chem. Res. 2020, 53, 4, 773-81; Dang et al., J. Org. Chem. 1996, 61, 5204-05), also react with 1,2,3-triazine 1-oxide 1a but they form the pyrazolo[1,5-a]pyrimidine isomer 11d in good yield whose atomic connectivity is clearly confirmed from its X-ray structure (Scheme 5). Surprisingly, only a trace of 11d is formed when 1,2,3-triazine 2a was treated with the 5-aminopyrrazole under the same conditions and for the same length of time; and 2a is recovered unchanged. Pyrazolo[ 1,5-a]pyrimidines form the core of a range of biologically active compounds (Kiessling et al., Chem. Med. Chem. 2007, 2, 627-30; Almansa et al., J. Med. Chem. 2001, 44, 350-61; Hwang et al., Bioorg. Med. Chem. Lett. 2012, 22, 7297-7301; Compton et al., J. Med. Chem. 2004, 47, 5872-93; Gana et al., Biochem. Pharmacol. 2019, 168, 237-48; Philoppes et al., Bioorg. Chem. 2020, 100, 103944-56), and their photophysical properties has made them a focus of study for promising new applications related to materials sciences (Tigreros et al., RSC Adv. 2020, 10, 39542-52; Singsardar et al., ChemistrySelect 2018, 3, 1404-10). They have been available by C-H functionalization (Bedford et al., Angew. Chem. Int. Ed. 2015, 54, 8787-90), from 1,3-dicarbonyl compounds, enaminones, or enones and 5-aminopyrrazole (Arias-Gomez et al., Molecules 2021, 26, 2708-43), and with other methodologies (Hammouda et al., RSC Med. Chem. 2022, 13, 1150-96; Salem et al., Synth. Commun. 2019, 49, 1750-76) each with their own limitations.

To determine the breadth of applications that form heterocyclic products using 1,2,3-triazine 1-oxides, we utilized a selection of derivatives of 1 with three different types of nucleophiles; and these results are reported in Schemes 3-5. Scheme 3 provides product yields from reactions with β-keto esters in DCM as the solvent and DBU as the base at room temperature. The reaction conditions used were optimized for solvent and base, and the relative amount of base could be lowered to 50 mol % without affecting product yield, but reaction times were lengthened (10 min with 2 equiv., 30 min with 1 equiv., and 2 h with 0.5 equiv. from 1a). All reported reactions with methyl 3-oxopentanoate and 2.0 equiv of DBU occurred within 30 min. The depicted yields in Scheme 3 were obtained from 0.1 mmol scale reactions. In addition, compound 4a was isolated in identical yield (92%) from a 1.0 mmol scale reaction. Electron-withdrawing groups decreased product yield, as did alkyl groups at the 5-position of the 1,2,3-triazine 1-oxide. Overall, however, product yields were consistently high. β-Keto esters directly derivatized from borneol, menthol, geraniol and cholesterol reacted at room temperature with triazine 1-oxide 1a to form the corresponding pyridine derivatives 4g-k in high yields showing potential late-stage functionalization. Analogues of these β-keto esters, dimethyl-3-oxoglutarate and methylsulfonylacetone also underwent IEDDA transformations in high isolated yield (Eq. 2 and 3) under the same conditions, further indicating the vast scope of pyridine syntheses from 1,2,3-triazine 1-oxides and β-keto compounds.

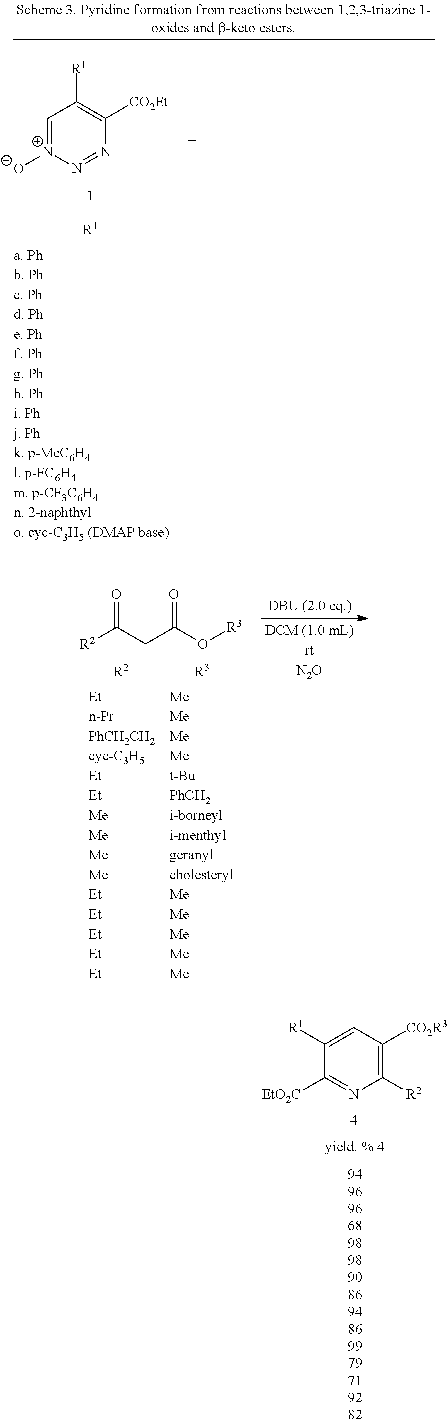

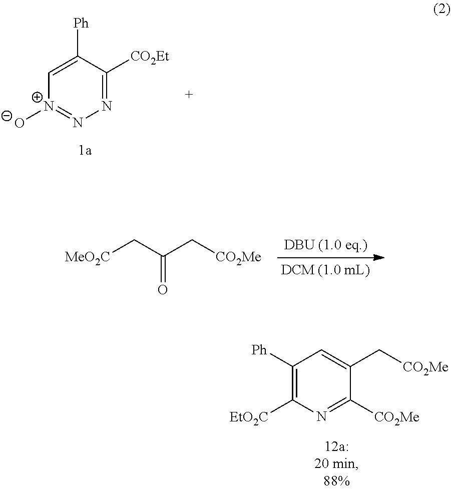 [SMILES: CCOC(=O)c1nn[n+]([O-])cc1-c1ccccc1 ; COC(=O)CC(=O)CC(=O)OC ; CCOC(=O)c1nc(C(=O)OC)c(CC(=O)OC)cc1-c1ccccc1]

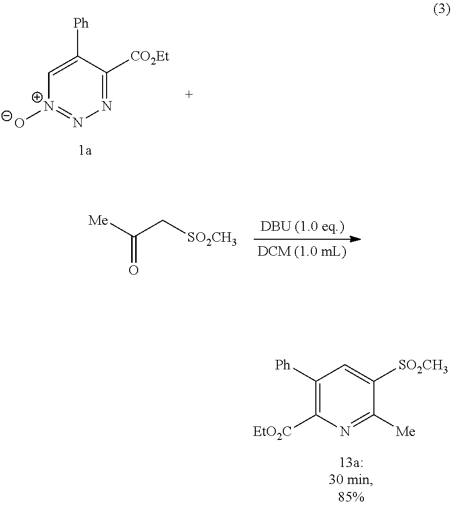 [SMILES: CCOC(=O)c1nn[n+]([O-])cc1-c1ccccc1 ; CC(=O)CS(C)(=O)=O ; CCOC(=O)c1nc(C)c(S(C)(=O)=O)cc1-c1ccccc1]

Scheme 4 reports results from the IEDDA reactions of 1 with dicyanomethane. Similar to results in Scheme 3, product yields were high, except for systems with electron-withdrawing groups or with alkyl groups at the 5-position. These reactions were very rapid at room temperature, much faster than those with beta-ketoesters, and generally occurred within five minutes.

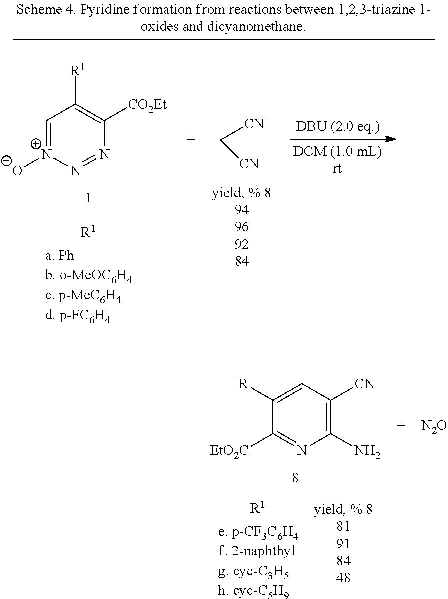

Scheme 5 contains the product outcome from reactions of representative 1,2,3-triazine 1-oxides 1 with representative 3/5-aminopyrazoles. Optimized conditions for solvent and base revealed that for IEDDA transformations with a longer reaction time than 30 minutes a weaker base than DBU is preferred because at the longer reaction times DBU caused destruction of the 1,2,3-triazine 1-oxide to a mixture of unidentified products. Pyrazolo[1,5-a]pyrimidine 11d was produced in 70% isolated yield in a solution of methylene chloride, triethylamine, triazine 1-oxide 1a and 3-(p-tolyl)-1H-pyrazol-5-amine in 72 h at room temperature. Interesting, under these mild basic reaction conditions, 1H-pyrazol-3-amine compounds undergo base catalyzed tautomerization to form 1H-pyrazol-5-amine compounds (Rios et al., Chemistry 2022, 4, 940-68) allowing the expansion of these IEDDA transformations with triazine 1-oxide 1 to more commercially available 1H-pyrazol-3-amine 1 derivatives to form pyrazolo[1,5-a]pyrimidines in high yields. As was observed in Scheme 3 and 4, product yields were high except for systems with electron-withdrawing groups at the 5-position of triazine 1-oxide 1.

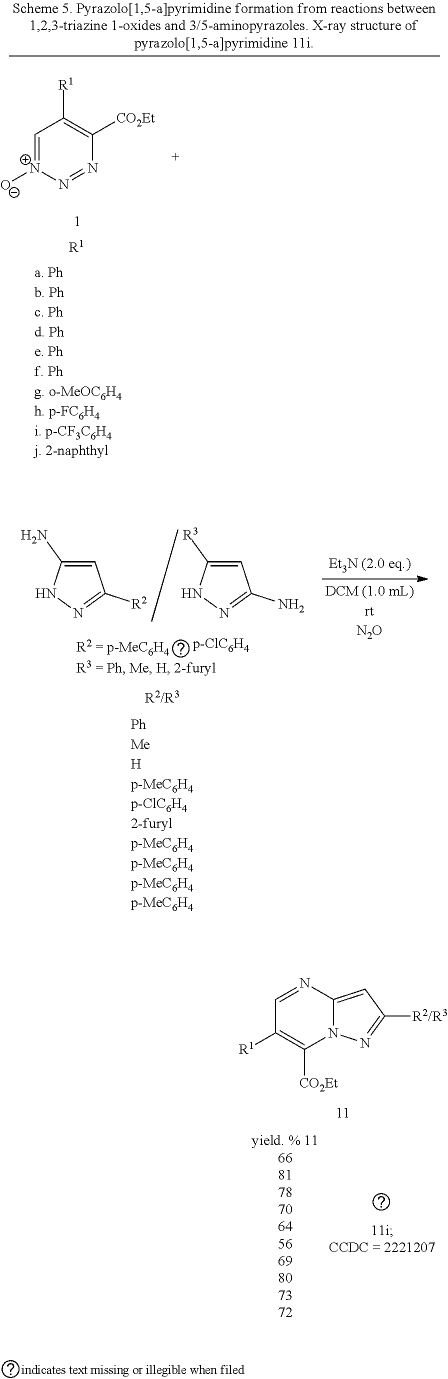

This composite methodology identifies 1,2,3-triazine 1-oxides as versatile substrates for the simple and practical synthesis of diverse N-heterocyclic compounds which are formed in high yields at room temperature without the use of catalysts, other than inexpensive bases, and with readily accessible reactants. These reactions occur by what is formally known as inverse electron demand Diels-Alder processes, initiated by nucleophilic attack at the 6-position of 1,2,3-triazines and 1,2,3-triazine 1-oxides, but their pathway can be by either a concerted or stepwise process. The broad generality of heterocyclic syntheses achieved with 1,2,3-triazine 1-oxides compliments or exceeds those achieved with 1,2,3-triazines and prompts investigations with nucleophiles other than those examined in this study. Ease of access to 1,2,3-triazine 1-oxides provides structures that are not readily accessible via traditional 1,2,3-triazine syntheses.

### I. 1,2,3-triazine 1-oxides

1,2,3-Triazine 1-oxides are a class of heterocyclic compounds containing a six-membered ring composed of three carbon atoms and three nitrogen atoms. The “1-oxides” designation indicates that there is an oxygen atom attached to one of the nitrogen atoms in the triazine ring forming an N-oxide functional group. These compounds find applications in various fields including organic synthesis, pharmaceuticals, and materials science due to their diverse chemical properties. They can serve as building blocks for the synthesis of more complex molecules or act as precursors for the preparation of functional materials.

The general structure of the 1,2,3-triazine 1-oxide reagents is provided in Formula I

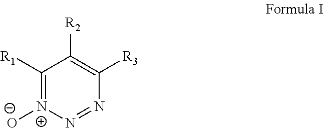

wherein R1 is hydrogen, R2 is aryl, polycyclic aryl, cyclopropyl, or functionalized alkyl without a H at the alpha position and R3 is an ester. In certain aspects the ester can be converted to other groups (e.g., CH₂OH, COOH, CONH₂, aldehyde, or ketone) or prepared as a sulfone or phosphonate.

In certain aspects R1 is hydrogen, and R2 is selected from substituted or unsubstituted cycloalkyl, substituted or unsubstituted heterocyclyl, substituted or unsubstituted monocyclic aryl, substituted or unsubstituted polycyclic aryl, functionalized alkyl without a H at the alpha position. and R3 is an ester. In certain aspects the ester can be converted to other groups (e.g., CH₂OH, COOH, CONH₂, aldehyde, or ketone) or prepared as a sulfone or phosphonate.

In certain aspects R1 is hydrogen, R2 is substituted or unsubstituted cycloalkyl, substituted or unsubstituted heterocyclyl, substituted or unsubstituted monocyclic aryl, substituted or unsubstituted polycyclic aryl, ester, or aldehyde. R3 is an ester. In one embodiment ester can be but is not limited to a methyl ester (—COOCH₃), ethyl ester (—COOCH₂CH₃), propyl ester (—COOCH₂CH₂CH₃), butyl ester (—COOCH₂CH₂CH₂CH₃), isopropyl ester (—COOCH(CH₃)₂), isobutyl ester (—COOCH(CH₃)CH₂CH₃), benzyl ester (—COOCH₂C₆Hs), or phenyl ester (—COO—C₆Hs). In certain aspects the ester can be converted to other groups (e.g., CH₂OH, COOH, CONH₂, aldehyde, or ketone) or prepared as a sulfone or phosphonate. More than 50 new 1,2,3-triazine 1-oxides have been prepared with different esters including terpenols and sterols indicating that there is no limitation to the types of esters used.

### II. Chemical Definitions

Various chemical definitions related to such compounds are provided as follows.

The term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxy” means —OH.

The term “alkyl,” by itself or as part of another substituent, means, unless otherwise stated, a linear (i.e. unbranched) or branched carbon chain, which may be fully saturated, mono-or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃(Me), —CH,CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃): (iso-butyl), —C(CH₃); (tert-butyl), —CH₂C(CH₃)₃ (neo-pentyl), are all non-limiting examples of alkyl groups.

The term “ester” means a chemical compound derived from an acid (organic or inorganic) in which at least one —OH (hydroxyl) group is replaced by an —O-alkyl (alkoxy) group. They are commonly formed by the reaction of carboxylic acids with alcohols. Ester groups include but are not limited to methyl ester (—COOCH₃), ethyl ester (—COOCH₂CH₃), propyl ester (—COOCH₂CH₂CH₃), butyl ester (—COOCH₂CH₂CH₂CH₃), isopropyl ester (—COOCH(CH₃)₂), isobutyl ester (—COOCH(CH₃)CH₂CH₃), benzyl ester (—COOCH₂C₆H₅), phenyl ester (—COO—C₆H₅), and the like. These are just a few examples of ester groups. Depending on the alkyl or aryl group attached to the carbonyl group, a wide variety of esters can be formed, each with its own distinct properties and applications.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂ Cl, —CH₂ Br, —CH₂ OH, —CH₂ OCH₃, —CH₂ OCH₂ CF₃, —CH₂OC(O)CH₃, —CH₂ NH₂, —CH₂ NHCH₃, —CH₂ N(CH₃)₂, —CH₂CH₂Cl, —CH₂CH₂OH, CH₂CH₂OC(O)CH₃, —CH₂CH₂ NHCO₂C(CH₃)₃, and —CH₂Si(CH₃)₃.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g. “C₁₋₄ alkylsulfonyl”).

### III. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Inverse Electron Demand Diels-Alder-Type Heterocycle Syntheses With 1,2,3-Triazine 1-oxides—Expanded Versatility

### Materials and Methods

All reactions, unless noted, were performed in oven-dried (150° C.) glassware with magnetic stirring under an atmosphere of air. Analytical thin layer chromatography (TLC) was carried out using EM Science silica gel 60 F254 plates; visualization was accomplished with UV light (254 nm). Column chromatography was performed on CombiFlash® Rf200 and Rf+ purification systems using normal phase disposable columns. NMR spectra were recorded on a on a Bruker spectrometer (500 MHz and 300 MHz) and calibrated using the resonance signal of the residual undeuterated solvent for ¹H-NMR [δ_{H}=7.26 ppm (CDCl₃)] and deuterated solvent for ¹³C-NMR [δ_{C}=77.16 (CDCl₃)] as an internal reference at 298 K. Spectra were reported as follows: chemical shift (8 ppm), multiplicity (Mi), coupling constants (Hz), integration and assignment. The peak information was described as: br=broad, s=singlet, d=doublet, t=triplet, q=quartet, dd=doublet of doublet, m=multiplet, and comp=composite of magnetically non-equivalent protons. ¹³C-NMR spectra were collected on Bruker instruments (126 MHz and 75 MHz) with complete proton decoupling. High-resolution mass spectra (HRMS) were performed on a Bruker MicroTOFESI mass spectrometer with an ESI resource using CsI or LTQ ESI positive ion calibration solution as the standard. Tetrahydrofuran, dichloromethane, chloroform, and toluene were purified using a JC-Meyer solvent purification system.

Materials: All B-keto-esters/carbonyls, malononitrile, hydrochloride salts of amidines, 3/5-aminopyrazoles, DBU (1,8-Diazabicyclo[5.4.0]undec-7-ene), DABCO (1,4-diazabicyclo[2.2.2]octane), DMAP (4-Dimethylaminopyridine), Et₃N (Triethylamine), C_{S}CO₃, K₂CO₃ were purchased from Sigma Aldrich, TCI, and Alfa Aesar, and were used without further purification. Amidine was prepared from its amidine hydrochloride salt by treating with equimolar sodium hydroxide. Beta keto esters derived from borneol, menthol, geraniol cholesterol was synthesized by reported procedure (Döben et al., Org. Lett. 2018, 20, 7933-36). Triazine-1-oxides and triazine were prepared by following the literature reported procedure (De Angelis et al., Org. Lett. 2021, 23, 6542-46; Rivera et al., Org. Lett. 2022, 24, 6543-47).

### Optimization of Reaction Conditions

TABLE 2
Optimization of reaction conditions for the IEDD cycloaddition between triazine 1-
oxide 1a and benzamidine.^{a}

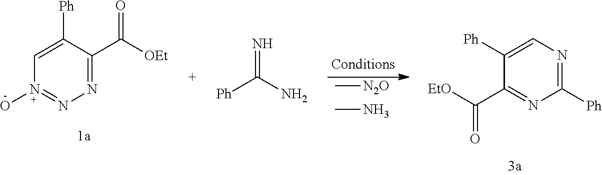

| | | Yield of 3a |
|---|---|---|
| Entry | Conditions | (%)^{b} |
| 1 | Dioxane, rt, <2 h | 97(95) |
| 2 | THF, rt, 2 h | 78 |
| 3 | CHCl₃, rt, 2 h | 64 |
| 4 | ACN, rt, 2 h | 71 |
| 5 | HFIP, rt, 2 h | <5%^{c} |
| 6 | DCM, rt, 2 h | 62^{d} |
| 7 | Tol, rt, 4 h | 68^{e} |
| 8 | Cyclohexane, rt, 4 h | 41^{f} |
| 9 | ACN, rt, 2 h | 31^{g} |

^{a}Reaction conditions: benzamidine was added to a solution of 1 (0.1 mmol in 1.0 mL) at room temperature.
^{b}NMR yields. Isolated yield in parenthesis.
^{c}90% of 1 was detected.
^{d}20% of 1 was detected.
^{e}22% of 1 was detected.
^{f}40% of 1 was detected.
^{g}35% of 1 was detected.

TABLE 3
Optimization of reaction conditions for the IEDD cycloaddition between triazine 1-
oxide 1a and methyl 3-oxopentanoate.^{a}

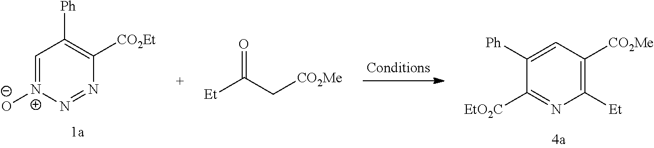 [SMILES: CCOC(=O)c1nn[n+]([O-])cc1-c1ccccc1 ; CCC(=O)CC(=O)OC ; CCOC(=O)c1nc(CC)c(C(=O)OC)cc1-c1ccccc1]

| Entry | Conditions | Base (2.0 equiv.) | Yield of 2a (%)^{b} |
|---|---|---|---|
| 1 | CHCl₃, rt, 24 h | Et₃N | 13 |
| 2 | CHCl₃, rt, 24 h | tBuOK | 64 |
| 3 | CHCl₃, rt, 24 h | K₂CO₃ | 83 |
| 4 | HFIP, rt, 24h | Cs₂CO₃ | 84 |
| 5 | CHCl₃, rt, 24 h | DMAP | 95 |
| 6 | CHCl₃, rt, 24 h | DABCO | 93 |
| 7 | CHCl₃, rt, 2 h | DBU | 97 |
| 8 | Dioxane, rt, 2 h | DBU | 71 |
| 9 | ACN, rt, 2h | DBU | 31 |
| 10 | DCM, rt, 10 min | DBU | 96(94) |
| 11^{c} | DCM, rt, 30 min | DBU | 96 |
| 12^{d} | DCM, rt, 2 h | DBU | 94 |

^{a}Reaction conditions: 1a (0.1 mmol) Methyl 3-oxopentanoate was added to a solution of at room temperature.
^{b}NMR yields. Isolated yield in parenthesis.
^{c}1.0 equiv. of DBU.
^{d}0.5 equiv. of DBU.

TABLE 4
Optimization of reaction conditions for the IEDD cycloadditions between triazine 1-
oxide 1a and 3-(p-tolyl)-1H-pyrazol-5-amine^{a}

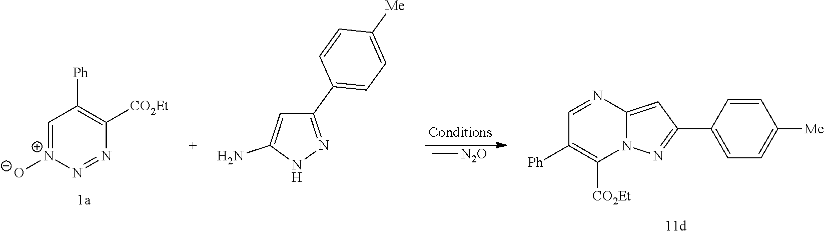

| | | | Yield of 11d |
|---|---|---|---|
| Entry | Conditions | Base (2.0 equiv.) | (%)^{b} |
| 1 | DCM, rt, 2 h | DBU | 8 |
| 2 | DCM, rt, 24 h | — | NR |
| 3 | DCM, rt, 24 h | K₂CO₃ | 15 |
| 4 | DCM, rt, 24 h | Cs₂CO₃ | <5 |
| 5 | DCM, rt, 72 h | DMAP | 65 |
| 6 | DCM, rt, 72 h | Et₃N | 72(70) |
| 7 | THF, rt, 72 h | Et₃N | 10 |
| 8 | ACN, rt, 72 h | Et₃N | 13 |
| 9 | DCM, 60° C., 72 h | Et₃N | 66 |
| 10 | ACN, 60° C., 72 h | Et₃N | 40 |
| 11 | ACN, 60° C., 72 h | DMAP | 35 |

^{a}Reaction conditions: 1a (0.1 mmol) was added to a solution of 3-(p-tolyl)-1H-pyrazol-5-amine (0.15 mmol) and Et₃N at room temperature.
^{b}NMR yields. Isolated yield in parenthesis.

General procedures for IEDDA reactions with triazine 1-oxide

Procedure 1: synthesis of pyrimidine compounds 3a-3k with triazine 1-oxide derivatives

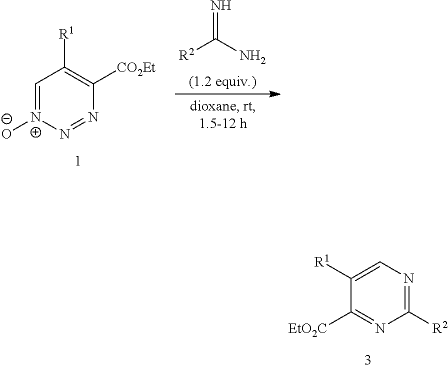

Triazine 1-oxide 1 (0.1 mmol) was dissolved in dioxane (0.5 mL) contained in a 7 mL vial. Amidine (1.2 equiv.), dissolved in dioxane (0.5 mL), was added at all once. The reaction was continued for 1.5 h at room temperature. After completion of the reaction (monitored by TLC) the solvent was removed under reduced pressure. Crude mass was transferred to silica gel column by dissolving minimal amount of dichloromethane and was purified by flash chromatography (% ethyl acetate in hexanes=20-40%) to give pyrimidine compounds 3 with good to excellent yields (66-95%)

Procedure 2: synthesis of pyridine compounds 4a-40 with triazine 1-oxide derivatives

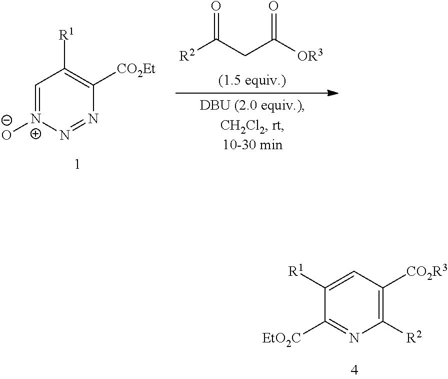

Triazine 1-oxide derivative 1 (0.1 mmol) was added all at once to a 1 mL solution containing β-keto ester (1.5 equiv.) and a DBU (2.0 equiv.). The reaction was continued for 10-30 minutes at room temperature. After the completion of the reaction (monitored by TLC), solvent was evaporated, and the product mixture was purified by flash chromatography (% ethyl acetate in hexanes=20-40%) to give the pyridine compound 4 with good to excellent yields (71-98%).

Procedure 3: synthesis of pyrazolo[1,5-a]pyrimidine compounds 11a-j with triazine 1-oxide derivatives

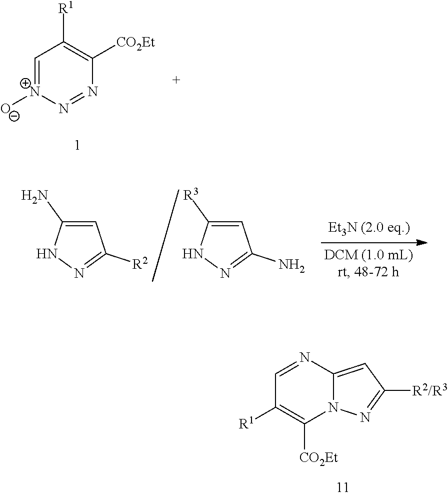

In a 1 mL solution of DCM containing amino pyrazole (1.5 equiv.) and Et₃N (2.0 equiv.), triazine 1-oxide 1 (0.1 mmol) was added all at once. The reaction was continued for 48-72 h at room temperature. After the completion of the reaction (monitored by TLC) solvent was evaporated, and the product mixture was purified directly by flash chromatography (% ethyl acetate in hexanes=30-50%) to give the pyrazolo[1,5-a]pyrimidine compounds 11 with moderate to good yields (56-81%).

Characterization of Nitrous oxide (N₂O) by IR spectroscopy in IEDDA reaction between 4-ethylcarboxylato-5-phenyl-1,2,3-triazine 1-oxide (1a) and ethyl 3-oxopentanoate.

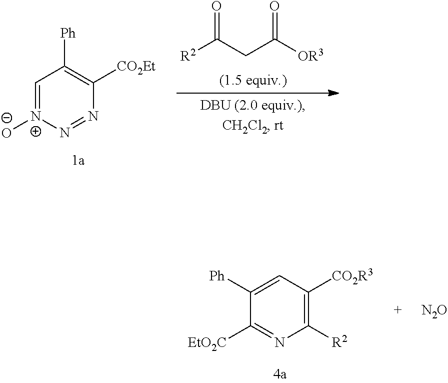

4-ethylcarboxylato-5-phenyl-1,2,3-triazine 1-oxide 1a (1.0 mmol) was dissolved in DCM (5 ml) in a 20 mL IR gas cell. The gas cell background IR was collected prior to addition of nucleophile to eliminate CO₂ signal. DCM (1.0 mL) containing ethyl 3-oxopentanoate (1.5 mmol) and DBU (2.0 mmol) was added to the gas cell at once. After 2 minutes, IR of the evolved gas was recorded. The obtained IR spectroscopy data appeared to be consistent with the literature reported one for Nitrous oxide (N₂O).⁴

Analytical and spectral characterization data for products

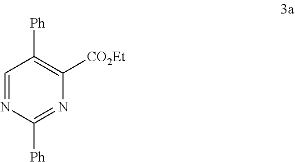 [SMILES: CCOC(=O)c1nc(-c2ccccc2)ncc1-c1ccccc1]

Ethyl 2,5-Diphenylpyrimidine-4-carboxylate, 3a; White solid, (28.8 mg, 96% yield), 0.1 mmol scale reaction. Flash column chromatography conditions: hexane:ethyl acetate=5:1. mp 66-68° C. ¹H NMR (500 MHZ, CDCl₃) δ 8.95 (s, 1H), 8.54-8.52 (comp, 2H), 7.52-7.7.40 (comp, 8H), 4.28 (q, J=7.1 Hz, 2H), 1.13 (t, J=7.1 Hz, 3H). ¹³C NMR (126 MHZ, CDCl₃) δ 165.9, 163.6, 158.9, 156.4, 136.7, 134.6, 131.3, 130.4, 129.0, 128.8, 128.8, 128.6, 128.6, 128.5, 62.23, 13.9. HRMS (ESI) calculated for [M+H]⁺ C₁₉H₁₆N₂O₂ m/z 305.1285, observed: 305.1288

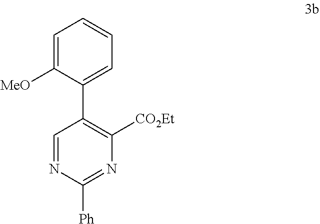 [SMILES: CCOC(=O)c1nc(-c2ccccc2)ncc1-c1ccccc1OC]

Ethyl 5-(2-Methoxyphenyl)-2-phenylpyrimidine-4-carboxylate, 3b. White solid, (30.1 mg, 90% yield), 0.1 mmol scale reaction. Flash column chromatography conditions: hexane:ethyl acetate=4:1. ¹H NMR (500 MHZ, CDCl₃) δ 8.92 (s, 1H), 8.52 (dd, J =6.7, 3.0 Hz, 2H), 7.58-7.47 (comp, 3H), 7.40-7.38 (m, 1H), 7.04-6.91 (comp, 3H), 4.30 (q, J=7.1 Hz, 2H), 3.85 (s, 3H), 1.17 (t, J=7.1 Hz, 3H). ¹³C NMR (126 MHZ, CDCl₃) δ 166.0, 163.6, 160.0, 158.8, 156.5, 136.7, 135.9, 131.3, 130.2, 130.1, 128.8, 128.6, 120.9, 114.4, 114.1, 62.3, 55.5, 13.9. HRMS (ESI) calculated for [M+H]+C₂₀H₁₈N₂O₃ m/z 335.1390, observed: 335.1391.

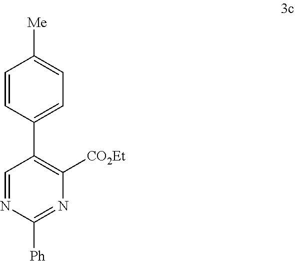 [SMILES: CCOC(=O)c1nc(-c2ccccc2)ncc1-c1ccc(C)cc1]

Ethyl 2-Phenyl-5-(p-tolyl)pyrimidine-4-carboxylate, 3c; White solid (29.6 mg, 92% yield), 0.1 mmol scale reaction. Flash column chromatography conditions: hexane:ethyl acetate=5:1. ¹H NMR (500 MHz, CDCl₃) δ 8.91 (s, 1H), 8.52 (dd, J=6.6, 3.1 Hz, 2H), 7.56-7.46 (comp, 3H), 7.35-7.26 (comp, 4H), 4.31 (q, J =7.2 Hz, 2H), 2.42 (s, 3H), 1.18 (t, J =7.2 Hz, 3H). ¹³C NMR (126 MHz, CDCl₃) δ166.01, 163.3, 158.9, 156.3, 138.9, 136.8, 131.6 131.2, 130.3, 129.7, 128.7, 128.5, 128.4, 62.2, 21.4, 13.9. HRMS (ESI) calculated for [M+H]⁺ C₂₀H₁₈N₂O₂ m/z 319.1441, observed: 319.1442.

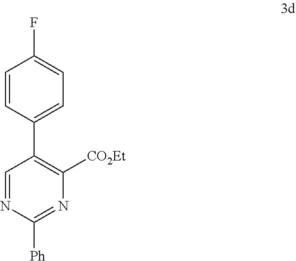 [SMILES: CCOC(=O)c1nc(-c2ccccc2)ncc1-c1ccc(F)cc1]

Ethyl 5-(4-Fluorophenyl)-2-phenylpyrimidine-4-carboxylate, 3d. White solid, (28.6 mg, 89% yield), 0.1 mmol scale reaction. Flash column chromatography conditions: hexane:ethyl acetate=5:1; ¹H NMR (500 MHZ, CDCl₃) δ 8.88 (s, 1H), 8.52-8.50 (comp, 2H), 7.53-7.50 (comp, 3H), 7.41-7.38 (comp, 2H), 7.19-7.14 (comp, 2H), 4.30 (q, J=7.2 Hz, 2H), 1.18 (t, J=7.2 Hz, 3H). ¹³C NMR (126 MHZ, CDCl₃) δ 165.8, 165.5 (d, J_{C-F}=250 Hz), 163.7, 162.3, 158.9, 156.3, 136.6, 131.4, 130.6 (d, J_{C-F}=4 Hz) , 130.4 (d, J_{C-F}=8.6 Hz), 129.5, 128.7 (d, J_{C-F}=22 Hz), 116.1 (d, J_{C-F}=22 Hz), 62.4, 14.0. HRMS (ESI) calculated for [M+H]⁺ C₁₉H₁₅FN₂O₂ m/z 323.1190, observed: 323.1191.

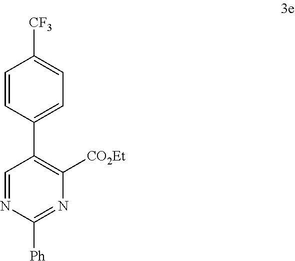 [SMILES: CCOC(=O)c1nc(-c2ccccc2)ncc1-c1ccc(C(F)(F)F)cc1]

Ethyl 2-Phenyl-5-(4-(trifluoromethyl)phenyl)pyrimidine-4-carboxylate, 3e. White solid, (33.8 mg, 91% yield), 0.1 mmol scale reaction. Flash column chromatography conditions: hexane:ethyl acetate=5:1. ¹H NMR (500 MHZ, CDCl₃) δ 8.90 (s, 1H), 8.56-8.52 (m, 2H), 7.75 (d, J=8.0 Hz, 2H), 7.55-7.47 (m, 5H), 4.30 (q, J=7.1 Hz, 2H), 1.16 (t, J=7.1 Hz, 3H). ¹³C NMR (126 MHz, CDCl₃) δ 165.4, 164.3, 158.8, 156.2, 138.5, 136.4, 131.6, 131.1 (q, J_{C-F}=32.5 Hz), 129.4, 129.0, 128.8, 128.7, 125.9 (q, J_{C-F}=4.0 Hz), 124.0 (q, J_{C-F}=272 Hz), 62.5, 13.9. HRMS (ESI) calculated for [M+H]+ C₂₀H₁₅F₃N₂O₂ m/z 373.1158, observed: 373.1160.

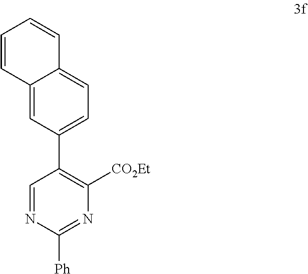 [SMILES: CCOC(=O)c1nc(-c2ccccc2)ncc1-c1ccc2ccccc2c1]

Ethyl 5-(Naphthalen-2-yl)-2-phenylpyrimidine-4-carboxylate, 3f. White solid, (31.2 mg, 88% yield), 0.1 mmol scale reaction. Flash column chromatography conditions: hexane:ethyl acetate=5:1.¹H NMR (500 MHZ, CDCl₃) δ 9.03 (s, 1H), 8.58-8.54 (comp, 2H), 7.96-7.89 (comp, 4H), 7.58-7.51 (comp, 6H), 4.27 (q, J=7.1 Hz, 2H), 1.09 (t, J=7.1 Hz, 3H). ¹³C NMR (126 MHz, CDCl₃) δ 166.0, 163.6, 159.2, 156.5, 136.7, 133.3, 133.1, 132.0, 131.3, 130.4, 128.8, 128.7, 128.6, 128.3, 127.9, 127.8, 127.0, 127.0, 126.0, 62.3, 13.9. HRMS (ESI) calculated for [M+H]⁺ C₂₃H₁₈N₂O₂ m/z 355.1441, observed: 355.1445.

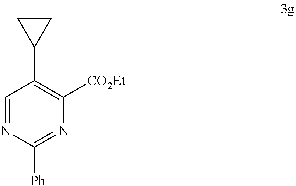 [SMILES: CCOC(=O)c1nc(-c2ccccc2)ncc1C1CC1]

Ethyl 5-Cyclopropyl-2-phenylpyrimidine-4-carboxylate, 3g. Viscous liquid, (26.9 mg, 71% yield), 0.1 mmol scale reaction. Flash column chromatography conditions: hexane:ethyl acetate=5:1. ¹H NMR (500 MHZ, CDCl₃) δ 8.57 (s, 1H), 8.44-8.41 (comp, 2H), 7.49-7.45 (comp, 3H), 4.52 (q, J=7.1 Hz, 2H), 2.29-2.26 (m, 1H), 1.47 (t, J=7.1 Hz, 3H), 1.12-1.08 (comp, 2H), 0.83-0.79 (comp, 2H). ¹³C NMR (126 MHz, CDCl₃) δ 165.9, 162.2, 157.3, 157.0, 137.0, 132.1, 130.9, 128.7, 128.3, 62.3, 14.4, 10.1, 8.0. HRMS (ESI) calculated for [M+H]⁺ C₁₆H₁₆N₂O₂ m/z 269.1285, observed: 269.1286.

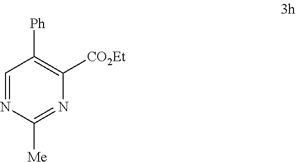 [SMILES: CCOC(=O)c1nc(C)ncc1-c1ccccc1]

Ethyl 2-Methyl-5-phenylpyrimidine-4-carboxylate, 3h. Viscous liquid, (16.0 mg, 66% yield), 0.1 mmol scale reaction. Flash column chromatography conditions: hexane:ethyl acetate=4:1. ¹H NMR (500 MHZ, CDCl₃) δ 8.76 (s, 1H), 7.46-7.42 (comp, 3H), 7.36-7.33 (comp, 2H), 4.23 (q, J=7.1 Hz, 2H), 2.84 (s, 3H), 1.10 (t, J=7.1 Hz, 3H). ¹³C NMR (126 MHZ, CDCl₃) δ 167.3, 165.7, 158.8, 155.9, 134.5, 130.0, 128.9, 128.8, 128.5, 62.3, 25.9, 13.8. HRMS (ESI) calculated for [M+H]⁺ C₁₄H₁₄N₂O₂ m/z 243.1128, observed: 243.1133.

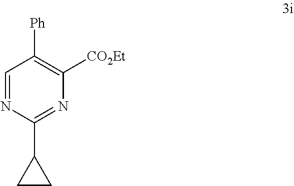 [SMILES: CCOC(=O)c1nc(C2CC2)ncc1-c1ccccc1]

Ethyl 2-Cyclopropyl-5-phenylpyrimidine-4-carboxylate, 3i. White solid, (23.8 mg, 89% yield), 0.1 mmol scale reaction. Flash column chromatography conditions: hexane:ethyl acetate=4:1.¹H NMR (500 MHZ, CDCl₃) δ 8.66 (s, 1H), 7.45-7.41 (comp, 3H), 7.34-7.32 (comp, 2H), 4.22 (q, J=7.1 Hz, 2H), 2.39-2.34 (comp, 1H), 1.24-1.21 (comp, 2H), 1.15-1.10 (comp, 2H), 1.09 (t, J=7.1 Hz, 3H). ¹³C NMR (126 MHZ, CDCl₃) δ 171.4, 166.0, 158.6, 155.8, 134.8, 129.4, 128.9, 128.6, 128.5, 62.2, 18.3, 13.8, 11.5. HRMS (ESI) calculated for [M+H]⁺ C₁₆H₁₆N₂O₂ m/z 269.1285, observed: 269.1291.

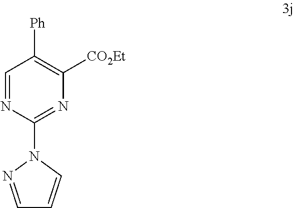 [SMILES: CCOC(=O)c1nc(-n2cccn2)ncc1-c1ccccc1]

Ethyl 5-Phenyl-2-(1H-pyrazol-1-yl)pyrimidine-4-carboxylate, 3j; White solid, (23.5 mg, 80% yield), 0.1 mmol scale reaction. Flash column chromatography conditions: hexane:ethyl acetate=3:1. ¹H NMR (500 MHZ, CDCl₃) δ 8.86 (s, 1H), 8.65-8.64 (m, 1H), 7.86 (s, 1H), 7.49-7.46 (comp, 3H), 7.40-7.38 (comp, 2H), 6.52 (s, 1H), 4.26 (q, J=7.1 Hz, 2H), 1.13 (t, J=7.1 Hz, 3H). ¹³C NMR (126 MHZ, CDCl₃) δ 164.8, 160.8, 157.7, 154.8, 144.3, 133.7, 130.4, 129.7, 129.1, 129.0, 129.0, 128.5, 109.2, 62.6, 13.8. HRMS (ESI) calculated for [M+H]⁺ C₁₆H₁₄N₄O₂ m/z 295.1190, observed: 295.1192.

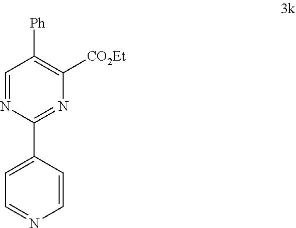 [SMILES: CCOC(=O)c1nc(-c2ccncc2)ncc1-c1ccccc1]

Ethyl 5-Phenyl-2-(pyridin-4-yl)pyrimidine-4-carboxylate, 3j; White solid, (26.8 mg, 88% yield), 0.1 mmol scale reaction; Flash column chromatography conditions: hexane:ethyl acetate=4:1;¹H NMR (500 MHZ, CDCl₃) δ 8.97 (s, 1H), 8.79 (d, J=5.0 Hz, 2H), 8.35 (d, J=5.0 Hz, 2H), 7.50-7.48 (comp, 3H), 7.43-7.41 (comp, 2H), 4.28 (q, J=7.1 Hz, 2H), 1.13 (t, J=7.1 Hz, 3H). ¹³C NMR (126 MHZ, CDCl₃) δ 165.5, 161.5, 159.1, 156.6, 150.6, 143.9, 134.0, 132.0, 129.2, 129.0, 128.5, 122.2, 62.4, 13.8. HRMS (ESI) calculated for [M+H]⁺ C₁₈H₁₅N₃O₂ m/z 306.1237, observed: 306.1242.

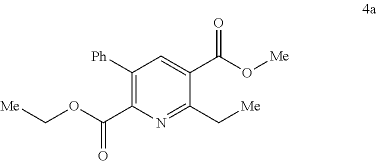 [SMILES: CCOC(=O)c1nc(CC)c(C(=O)OC)cc1-c1ccccc1]

Ethyl 5-Methylcarboxylato-6-ethyl-3-phenylpyridine-2-carboxylate, 4a. White solid, (29.4 mg, 94% yield), 0.1 mmol scale reaction. Flash column chromatography conditions: hexane:ethyl acetate=5:1. mp 52-54° C. ¹H NMR (500 MHZ, CDCl₃) δ 8.21 (s, 1H), 7.46-7.39 (comp, 3H), 7.38-7.34 (comp, 2H), 4.19 (q, J=7.1 Hz, 2H), 3.94 (s, 3H), 3.24 (q, J=7.5 Hz, 2H), 1.35 (t, J=7.5 Hz, 2H), 1.05 (t, J=7.1 Hz, 3H). ¹³C NMR (126 MHZ, CDCl₃) δ 167.0, 166.5, 163.3, 151.3, 140.5, 137.3, 133.7, 128.7, 128.4, 128.3, 126.2, 61.9, 52.6, 30.1, 14.2, 13.7. HRMS (ESI) calculated for [M+H]⁺ C₁₈H₁₉NO₄ m/z 314.1387, observed: 314.1386.

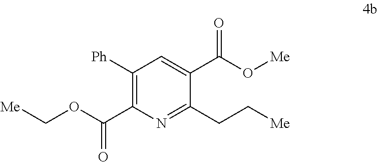 [SMILES: CCCc1nc(C(=O)OCC)c(-c2ccccc2)cc1C(=O)OC]

Ethyl 5-Methylcarboxylato-3-phenyl-6-propylpyridine-2-carboxylate, 4b. Viscous liquid, (31.4 mg, 96% yield), 0.1 mmol scale reaction. Flash column chromatography conditions: hexane:ethyl acetate=5:1. ¹H NMR (500 MHZ, CDCl₃) δ 8.21 (s, 1H), 7.40-7.44 (comp, 3H), 7.39-7.35 (comp, 2H), 4.19 (q, J=7.1 Hz, 2H), 3.93 (s, 3H), 3.21-3.18 (m, 2H), 1.78 (sext, J=7.7 Hz, 2H), 1.05-1.01 (comp, 6H). ¹³C NMR (126 MHZ, CDCl₃) δ 167.0, 166.5, 162.2, 151.3, 140.5, 137.3, 133.7, 128.7, 128.4, 128.3, 126.5, 61.9, 52.7, 38.6, 23.5, 14.4, 13.8. HRMS (ESI) calculated for [M+H]⁺ C₁₉H₂₁NO₄ m/z 328.1543, observed: 328.1549.

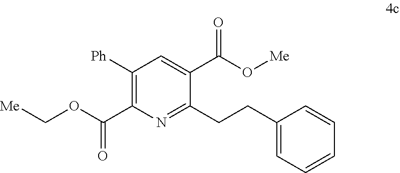 [SMILES: CCOC(=O)c1nc(CCc2ccccc2)c(C(=O)OC)cc1-c1ccccc1]

Ethyl 5-Methylcarboxylato-6-phenethyl-3-phenylpyridine-2-carboxylate, 4c. Viscous liquid, (37.3 mg, 96% yield), 0.1 mmol scale reaction. Flash column chromatography conditions: hexane:ethyl acetate=5:1.¹H NMR (500 MHZ, CDCl³) δ 8.25 (s, 1H), 7.46-7.41 (comp, 3H), 7.40-7.37 (comp, 2H), 7.35-7.28 (comp, 4H), 7.22-7.19 (comp, 1H), 4.22 (q, J=7.2 Hz, 2H), 3.92 (s, 3H), 3.57-3.53 (comp, 2H), 3.11-3.07 (comp, 2H), 1.07 (t, J=7.1 Hz, 3H). ¹³C NMR (126 MHz, CDCl³) δ 167.0, 166.2, 161.1, 151.4, 141.9, 140.6, 137.2, 134.0, 128.8, 128.7, 128.5, 128.4, 126.5, 126.0, 62.0, 52.7, 38.6, 36.0, 13.8. HRMS (ESI) calculated for [M+H]⁺ C²⁴H²³NO⁴ m/z 390.1700, observed: 390.1704.

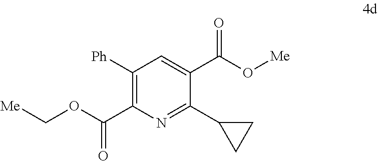 [SMILES: CCOC(=O)c1nc(C2CC2)c(C(=O)OC)cc1-c1ccccc1]

Ethyl 5-Methylcarboxylato-6-cyclopropyl-3-phenylpyridine-2-carboxylate, 4d. White solid, (22.1 mg, 68% yield), 0.1 mmol scale reaction. Flash column chromatography conditions: hexane:ethyl acetate=5:1. ¹H NMR (500 MHZ, CDCl₃) δ 8.15 (s, 1H), 7.42-7.32 (comp, 5H), 4.16 (q, J=7.1 Hz, 2H), 3.95 (s, 3H), 3.05 (tt, J=8.4, 4.8 Hz, 1H), 1.09-1.00 (comp, 5H), 0.90-0.82 (comp, 2H). ¹³C NMR (126 MHz, CDCl₃) δ 167.1, 167.0, 162.5, 151.5, 140.0, 137.4, 132.2, 128.7, 128.4, 128.2, 126.1, 61.7, 52.6, 14.4, 13.8, 11.6. HRMS (ESI) calculated for [M+H]⁺ C₁₉H₁₉NO₄ m/z 326.1387, observed: 326.1391.

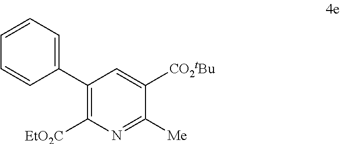 [SMILES: CCOC(=O)c1nc(C)c(C(=O)OC(C)(C)C)cc1-c1ccccc1]

tert-Butyl 2-Ethylcarboxylato-6-methyl-3-phenylpyridine-5-carboxylate, 4e. White solid, (33.4 mg, 98% yield), 0.1 mmol scale reaction. Flash column chromatography conditions: hexane:ethyl acetate=5:1. ¹H NMR (500 MHZ, CDCl₃) δ 8.14 (s, 1H), 7.44-7.39 (comp, 3H), 7.36-7.34 (comp, 2H), 4.18 (q, J=7.1 Hz, 2H), 2.86 (s, 3H), 1.60 (s, 9H), 1.04 (t, J =7.1 Hz, 3H). ¹³C NMR (126 MHZ, CDCl₃) δ 166.9, 165.4, 157.8, 150.4, 140.2, 137.5, 134.0, 128.6, 128.6, 128.4, 128.2, 82.7, 61.8, 28.3, 24.6, 13.7. HRMS (ESI) calculated for [M+H]⁺ C₂₀H₂₃NO₄ m/z 342.1700, observed: 342.1705.

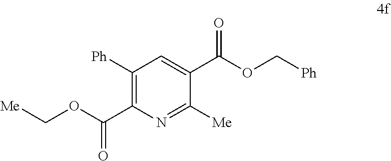 [SMILES: CCOC(=O)c1nc(C)c(C(=O)OCc2ccccc2)cc1-c1ccccc1]

Benzyl 2-Ethylcarboxylato-6-methyl-3-phenylpyridine-5-carboxylate, 4f. White solid, (39.0 mg, 98% yield), 0.1 mmol scale reaction. Flash column chromatography conditions: hexane:ethyl acetate=5:1.¹H NMR (500 MHZ, CDCl₃) δ 8.27 (s, 1H), 7.46-7.32 (comp, 10H), 5.38 (s, 2H), 4.19 (q, J=7.1 Hz, 2H), 2.91 (s, 3H), 1.05 (t, J=7.1 Hz, 3H). ¹³C NMR (126 MHz, CDCl₃) δ 166.8, 165.7, 158.7, 151.1, 140.5, 137.2, 135.4, 134.0, 128.8, 128.7, 128.7, 129.0, 128.4, 128.3, 126.5, 67.5, 62.0, 24.6, 13.8. HRMS (ESI) calculated for [M+H]⁺ C₂₃H₂₁NO₄ m/z 376.1543, observed: 376.1552.

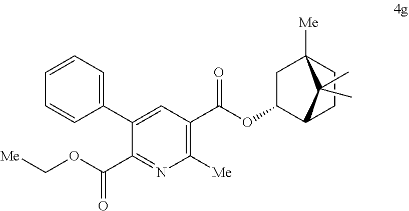

Ethyl 5-((1R,2R,4S)-4,7,7-Trimethylbicyclo[2.2.1]heptan-2-yl)carboxylato-6-methyl-3-phenylpyridine-2-carboxylate, 4g. Viscous liquid, (37.9 mg, 90% yield), 0.1 mmol scale reaction. Flash column chromatography conditions: hexane:ethyl acetate=5:1. ¹H NMR (500 MHZ, CDCl₃) δ 8.22 (s, 1H), 7.42 (dd, J=9.5, 6.8 Hz, 3H), 7.40-7.35 (comp, 2H), 5.15 (dt, J=9.9, 2.8 Hz, 1H), 4.19 (q, J=7.1 Hz, 2H), 2.91 (s, 3H), 2.58-2.44 (m, 1H), 1.99 (ddd, J=13.4, 9.4, 4.3 Hz, 1H), 1.77 (dq, J=22.1, 4.3 Hz, 2H), 1.46-1.34 (m, 1H), 1.33-1.24 (m, 1H), 1.12 (dd, J=13.9, 3.5 Hz, 1H), 1.05 (t, J=7.1 Hz, 3H), 0.97 (s, 3H), 0.92 (s,, 3H), 0.90 (s, 3H). ¹³C NMR (126 MHZ, CDCl₃) δ 166.8, 166.5, 158.1, 150.9, 140.2, 137.4, 134.0, 128.7, 128.4, 128.3, 127.4, 82.0, 61.9, 49.1, 48.1, 45.0, 37.1, 28.2, 27.6, 24.7, 19.8, 19.0, 13.8, 13.8. HRMS (ESI) calculated for [M+H]⁺ C₂₆H₃₁NO₄ m/z 422.2326, observed: 422.2327.

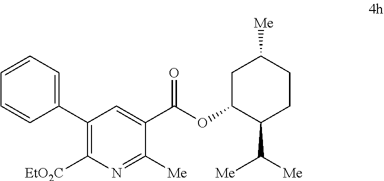 [SMILES: CCOC(=O)c1nc(C)c(C(=O)O[C@@H]2C[C@H](C)CC[C@H]2C(C)C)cc1-c1ccccc1]

Ethyl 5-((1R,2S,5R)-2-Isopropyl-5-methylcyclohexyl)carboxylato-6-methyl-3-phenylpyridine-2-carboxylate, 4h. Viscous liquid, (36.4 mg, 86% yield), 0.1 mmol scale reaction. Flash column chromatography conditions: hexane:ethyl acetate=5:1. ¹H NMR (500 MHz, CDCl₃) δ 8.20 (s, 1H), 7.46-7.40 (comp, 3H), 7.38-7.35 (comp, 2H), 4.99 (td, J=10.9, 4.3 Hz, 1H), 4.18 (q, J=7.1Hz, 2H), 2.89 (s, 3H), 2.12 (dt, J=12.1, 4.0 Hz, 1H), 1.89 (pd, J=7.0, 2.3 Hz, 1H), 1.71-1.75 (m, 3H), 1.62-1.49 (comp, 2H), 1.13 (q, J=11.9 Hz, 2H), 1.04 (t, J=7.1 Hz, 3H), 0.93 (d, J=7.1 Hz, 3H), 0.90 (d, J=7.1 Hz, 3H), 0.80 (d, J=7.0 Hz, 3H). ¹³C NMR (126 MHZ, CDCl₃) δ 166.9, 165.6, 158.3, 150.8, 140.2, 137.4, 134.0, 128.7, 128.4, 128.3, 127.4, 75.9, 61.9, 47.2, 41.1, 34.3, 31.6, 26.6, 24.6, 23.6, 22.1, 20.9, 16.5, 13.8. HRMS (ESI) calculated for [M+H]⁺ C₂₆H₃₃NO₄ m/z 424.2482, observed: 424.2490.

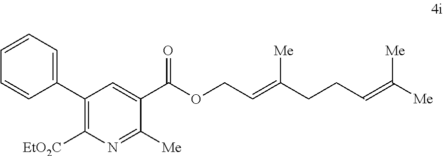 [SMILES: CCOC(=O)c1nc(C)c(C(=O)OC/C=C(\C)CCC=C(C)C)cc1-c1ccccc1]

(E)-5-(3,7-dimethylocta-2,6-dien-1-yl) 2-ethyl 6-methyl-3-phenylpyridine-2,5-dicarboxylate, 4i. Gummy liquid, (39.5 mg, 94% yield), 0.1 mmol scale reaction. Flash column chromatography conditions:hexane:ethyl acetate=5:1.¹H NMR (500 MHZ, CDCl₃) δ 8.24 (s, 1H), 7.44-7.39 (comp, 3H), 7.36-7.34 (comp, 2H), 5.45 (t, J=7.3 Hz, 1H), 5.08-5.05 (m, 1H), 4.86 (d, J=7.1 Hz, 2H), 4.19 (q, J=7.1 Hz, 2H), 2.90 (s, 3H), 2.12-2.07 (comp, 4H), 1.76 (s, 3H), 1.65 (s, 3H), 1.59 (s, 3H), 1.09-1.04 (d, J=7.1 Hz, 3H). ¹³C NMR (126 MHZ, CDCl₃) δ 166.9, 166.0, 158.5, 150.9, 143.309, 140.5, 137.4, 134.0, 132.0, 128.6, 128.4, 128.3, 127.0, 123.7, 117.91, 62.6, 61.9, 39.6, 26.3, 25.8, 24.6, 17.8, 16.7, 13.8. HRMS (ESI) calculated for [M+H]⁺ C₂₆H₃₁NO₄ m/z 422.2326, observed: 422.2333.

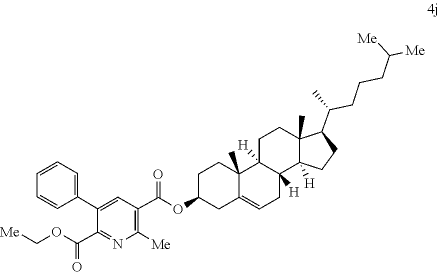 [SMILES: CCOC(=O)c1nc(C)c(C(=O)O[C@H]2CC[C@@]3(C)C(=CC[C@H]4[C@@H]5CC[C@H]([C@H](C)CCCC(C)C)[C@@]5(C)CC[C@@H]43)C2)cc1-c1ccccc1]

5-[(3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)]2-ethyl 6-methyl-3-phenylpyridine-2,5-dicarboxylate, 4j. Viscous liquid, (56.1 mg, 86% yield), 0.1 mmol scale reaction. Flash column chromatography conditions: hexane:ethyl acetate=5:1. 1H NMR (500 MHZ, CDCl₃) δ 8.22 (s, 1H), 7.46-7.39 (comp, 3H), 7.36 (dd, J=7.8, 1.8 Hz, 2H), 5.42 (d, J=4.9 Hz, 1H), 4.89 (dtt, J=11.4, 7.6, 4.5 Hz, 1H), 4.18 (q, J=7.2 Hz, 2H), 2.90 (s, 3H), 2.46 (d, J=8.0 Hz, 2H), 2.00 (tq, J=13.4, 5.0, 4.3 Hz, 3H), 1.92 (dt, J=13.5, 3.6 Hz, 1H), 1.83 (ddt, J=13.1, 9.5, 5.9 Hz, 1H), 1.80-1.66 (m, 1H), 1.63-1.42 (comp, 6H), 1.41-0.94 (comp, 20H), 0.92 (d, J=6.4 Hz, 3H), 0.86 (dd, J=6.6, 2.3 Hz, 6H), 0.68 (s, 3H). ¹³C NMR (126 MHZ, CDCl₃) δ 166.8, 165.4, 158.3, 150.8, 140.3, 139.4, 137.4, 134.0, 128.6, 128.4, 128.2, 127.2, 123.2, 75.6, 61.9, 56.3, 56.2, 50.1, 42.4, 39.8, 39.6, 38.2, 37.1, 36.7, 36.2, 35.9, 34.7, 32.0, 32.0, 28.3, 28.1, 28.0, 27.9, 25.3, 24.6, 24.4, 23.9, 22.9, 22.7, 21.1, 19.4, 18.8, 13.7, 12.0. HRMS (ESI) calculated for [M+H]⁺ C₄₃H₅₉NO₄ m/z 654.4517, observed: 654.4523.

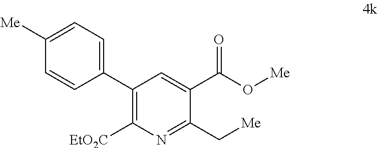 [SMILES: CCOC(=O)c1nc(CC)c(C(=O)OC)cc1-c1ccc(C)cc1]

Ethyl 5-Methylcarboxylato-6-ethyl-3-(p-tolyl)pyridine-2-carboxylate, 4k. White solid, (32.4 mg, 99% yield), 0.1 mmol scale reaction. Flash column chromatography conditions: hexane:ethyl acetate=5:1. ¹H NMR (500 MHZ, CDCl₃) δ 8.22 (s, 1H), 7.29-7.23 (comp, 4H), 4.24 (q, J=7.1, 2H), 3.95 (s, 3H), 3.25 (q, J=7.5, 2H), 2.41 (s, 3H), 1.36 (t, J=7.5, 3H), 1.12 (t, J=7.1, 3H). ¹³C NMR (126 MHZ, CDCl₃) δ 167.1, 166.5, 163.0, 151.3, 140.5, 138.2, 134.3, 133.6, 129.4, 128.2, 126.2, 61.9, 52.6, 30.0, 21.3, 14.2, 13.8. HRMS (ESI) calculated for [M+H]⁺ C₁₉H₂₁NO₄ m/z 323.1543, observed: 328.1548.

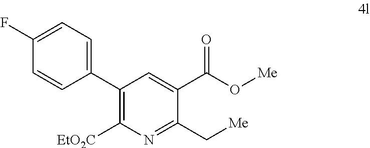 [SMILES: CCOC(=O)c1nc(CC)c(C(=O)OC)cc1-c1ccc(F)cc1]

Ethyl 5-Methylcarboxylato-6-ethyl-3-(4-fluorophenyl)pyridine-2-carboxylate, 41. White solid, (26.1 mg, 79% yield), 0.1 mmol scale reaction. Flash column chromatography conditions: hexane:ethyl acetate=5:1.¹H NMR (500 MHZ, CDCl₃) δ 8.17 (s, 1H), 7.35-7.32 (comp, 2H), 7.12 (t, J=8.4 Hz, 2H), 4.21 (q, J=7.1, 2H), 3.94 (s, 3H), 3.23 (q, J=7.5 Hz, 2H), 1.34 (t, J=7.5 Hz, 3H), 1.10 (t, J=7.1, 3H). ¹³C NMR (126 MHz, CDCl₃) δ 166.8, 166.4, 163.4, 163.0 (d, J_{C-F}=248 Hz) 151.2, 140.5, 133.4, 133.4, 132.7, 130.2, 130.2, 126.3, 115.7 (d, JC-F=21 Hz), 62.0, 52.7, 30.1, 14.1, 13.9. HRMS (ESI) calculated for [M+H]⁺ C₁₈H₁₈FNO₄ m/z 332.1293, observed: 332.1297.

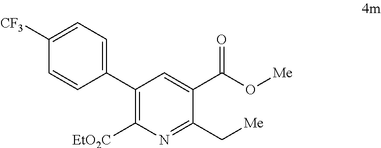 [SMILES: CCOC(=O)c1nc(CC)c(C(=O)OC)cc1-c1ccc(C(F)(F)F)cc1]

Ethyl 5-Methylcarboxylato-6-ethyl-3-(4-(trifluoromethyl)phenyl)pyridine-2-carboxylate, 4m. White solid, (27.1 mg, 71% yield), 0.1 mmol scale reaction. Flash column chromatography conditions: hexane:ethyl acetate=5:1. ¹H NMR (500 MHZ, CDCl₃) δ 8.20 (s, 1H), 7.69 (d, J=7.9 Hz, 2H), 7.48 (d, J=7.9 Hz, 2H), 4.21 (q, J=7.1 Hz, 2H), 3.95 (s, 3H), 3.26 (q, J=7.5 Hz, 2H), 1.35 (t, J=7.5 Hz, 3H), 1.07 (t, J=7.1 Hz, 3H). ¹³C NMR (126 MHZ, CDCl₃) δ 166.4, 166.2, 164.1, 150.9, 141.2, 140.5, 132.6, 130.5 (q, J_{C-F}=32.0 Hz), 128.9, 126.4, 125.6 (q, J_{C-F}=4.0 Hz), 124.1 (q, J_{C-F}=270.0 Hz), 62.1, 52.79, 30.15, 14.10, 13.77. HRMS (ESI) calculated for [M+H]+C₁₉H₁₈F₃NO₄ m/z 382.1261, observed: 382.1267.

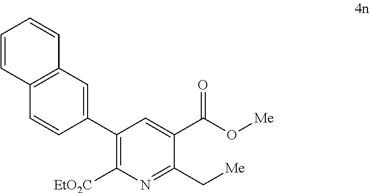 [SMILES: CCOC(=O)c1nc(CC)c(C(=O)OC)cc1-c1ccc2ccccc2c1]

Ethyl 5-Methylcarboxylato-6-ethyl-3-(naphthalen-2-yl)pyridine-2- carboxylate, 4n. White solid, (33.4 mg, 92% yield), 0.1 mmol scale reaction. Flash column chromatography conditions: hexane:ethyl acetate=5:1. ¹H NMR (500 MHZ, CDCl₃) δ 8.32 (s, 1H), 7.91-7.85 (comp, 4H), 7.54-7.47 (comp, 3H), 4.18 (q, J=7.1 Hz, 2H), 3.95 (s, 3H), 3.28 (q, J=7.5 Hz, 2H) 1.37 (t, J=7.5 Hz, 3H), 0.98 (t, J=7.1 Hz, 3H). ¹³C NMR (126 MHZ, CDCl₃) δ 167.0, 166.5, 163.3, 151.4, 140.8, 134.7, 133.7, 133.3, 132.9, 128.4, 128.2, 127.9, 127.6, 126.8, 126.7, 126.3, 126.2, 62.0, 52.7, 30.1, 14.2, 13.82. HRMS (ESI) calculated for [M+H]⁺ C₂₂H₂₁NO₄ m/z 364.1543, observed: 364.1550.

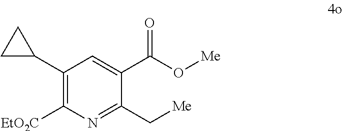 [SMILES: CCOC(=O)c1nc(CC)c(C(=O)OC)cc1C1CC1]

Ethyl 5-Methylcarboxylato-3-cyclopropyl-6-ethylpyridine-2- carboxylate, 40. White solid, (22.7 mg, 82% yield), 0.1 mmol scale reaction. Flash column chromatography conditions: hexane:ethyl acetate=5:1. ¹H NMR (500 MHZ, CDCl₃) δ 7.76 (s, 1H), 4.47 (q, J=7.1 Hz, 2H), 3.91 (s, 3H), 3.13 (q, J=7.5 Hz, 2H), 2.26 (ddd, J=13.7, 8.5, 5.2 Hz, 1H), 1.42 (t, J=7.1 Hz, 3H), 1.26 (t, J=7.5 Hz, 3H), 1.04-0.98 (comp, 2H), 0.72-0.67 (comp, 2H). ¹³C NMR (126 MHz, CDCl₃) δ 166.9, 166.7, 161.3, 152.4, 136.8, 135.2, 126.4, 62.0, 52.6, 29.9, 14.4, 14.2, 11.6, 8.3. HRMS (ESI) calculated for [M+H]⁺ C₁₅H₁₉NO₄ m/z 278.1387, observed: 278.1392.

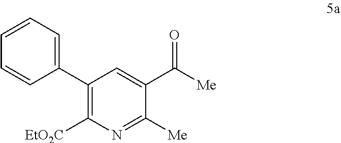 [SMILES: CCOC(=O)c1nc(C)c(C(C)=O)cc1-c1ccccc1]

Ethyl 5-Acetyl-6-methyl-3-phenylpicolinate, 5a. Viscous liquid, (17.6 mg, 62% yield), 0.1 mmol scale reaction. Flash column chromatography conditions: hexane:ethyl acetate=5:1. ¹H NMR (500 MHZ, CDCl₃) δ 7.97 (s, 1H), 7.44-7.41 (comp, 3H), 7.37-7.35 (comp, 2H), 4.19 (q, J=7.1 Hz, 2H), 2.81 (s, 3H), 2.62 (s, 3H), 1.05 (t, J=7.1 Hz, 3H). ¹³C NMR (126 MHz, CDCl₃) δ 200.0, 166.7, 156.8, 150.4, 138.7, 137.4, 134.2, 134.0, 128.8, 128.4, 128.4, 62.0, 29.7, 24.4, 13.8. HRMS (ESI) calculated for [M+H]⁺ C₁₇H₁₇NO₃ m/z 284.1281, observed: 284.1284.

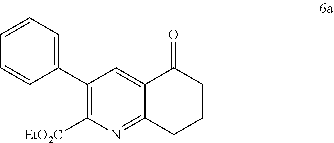 [SMILES: CCOC(=O)c1nc2c(cc1-c1ccccc1)C(=O)CCC2]

Ethyl 5-Oxo-3-phenyl-5,6,7,8-tetrahydroquinoline-2-carboxylate, 6a. White solid, (23.6 mg, 80% yield), 0.1 mmol scale reaction. Flash column chromatography conditions: hexane:ethyl acetate=5:1. mp 75-77° C. ¹H NMR (500 MHZ, CDCl₃) δ 8.34 (s, 1H), 7.45-7.38 (comp, 3H), 7.36-7.34 (comp, 2H), 4.20 (q, J=7.1 Hz, 2H), 3.25 (t, J=6.2 Hz, 2H), 2.76-2.73 (comp, 2H), 2.25-2.22 (comp, 2H), 1.05 (t, J=7.1 Hz, 3H). ¹³C NMR (126 MHZ, CDCl₃) δ 197.4, 166.9, 162.1, 152.7, 137.2, 137.0, 135.2, 128.7, 128.7, 128.4, 128.4, 62.1, 38.7, 32.2, 21.8, 13.8. HRMS (ESI) calculated for [M+H]^{+ β}C₁₈H₁₇NO₃ m/z 296.1281, observed: 296.1284.

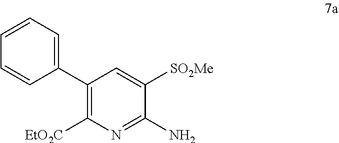 [SMILES: CCOC(=O)c1nc(N)c(S(C)(=O)=O)cc1-c1ccccc1]

Ethyl 6-Amino-5-(methylsulfonyl)-3-phenylpicolinate, 7a. White solid, (26.9 mg, 84% yield), 0.1 mmol scale reaction. Flash column chromatography conditions: hexane:ethyl acetate=5:1. mp 172-174° C. ¹H NMR (500 MHZ, CDCl₃) δ 8.10 (s, 1H), 7.42-7.37 (comp, 3H), 7.31-7.30 (comp, 2H), 5.99 (br s, 2H), 4.17 (q, J=7.1 Hz, 2H), 3.11 (s, 3H), 1.04 (t, J=7.1 Hz, 3H). ¹³C NMR (126 MHZ, CDCl₃) δ 166.3, 154.4, 153.1, 141.2, 136.7, 128.8, 128.3, 128.2, 127.3, 118.9, 62.2, 42.6, 13.8. HRMS (ESI) calculated for [M+H]⁺ C₁₅H₁₆N₂O₄S m/z 321.0904, observed: 321.0910.

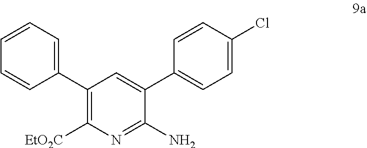 [SMILES: CCOC(=O)c1nc(N)c(-c2ccc(Cl)cc2)cc1-c1ccccc1]

Ethyl 6-Amino-5-(4-chlorophenyl)-3-phenylpicolinate, 9a. White solid, (17.6 mg, 50% yield), 0.1 mmol scale reaction. Flash column chromatography conditions: hexane:ethyl acetate=5:1. mp 98-100° C. ¹H NMR (500 MHZ, CDCl₃) δ 7.46-7.42 (comp, 5H), 7.40-7.36 (comp, 2H), 7.3-7.31 (comp, 3H), 4.85 (br s, 2H), 4.16 (q, J=7.1 Hz, 2H), 1.04 (t, J=7.1 Hz, 3H). ¹³C NMR (126 MHZ, CDCl₃) δ 167.3, 154.6, 146.3, 140.2, 138.7, 135.4, 134.6, 130.1, 129.6, 128.4, 128.4, 127.4, 122.8, 61.6, 13.8. HRMS (ESI) calculated for [M+H]⁺ C₂₀H₁₇ClN₂O₂ m/z 353.1051, observed: 353.1052.

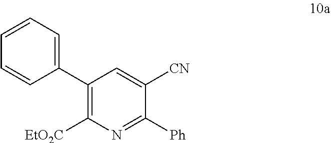 [SMILES: CCOC(=O)c1nc(-c2ccccc2)c(C#N)cc1-c1ccccc1]

Ethyl 5-Cyano-3,6-diphenylpicolinate, 10a. White solid, (20.0 mg, 61% yield), 0.1 mmol scale reaction. Flash column chromatography conditions: hexane:ethyl acetate=5:1. mp 70-72° C. ¹H NMR (500 MHZ, CDCl₃) δ 8.15 (s, 1H), 8.03-8.00 (comp, 2H), 7.55-7.53 (comp, 3H), 7.48-7.47 (comp, 3H), 7.42-7.40 (m, 2H), 4.23 (q, J=7.1 Hz, 2H), 1.09 (t, J=7.1 Hz, 3H). ¹³C NMR (126 MHZ, CDCl₃) δ 166.1, 159.1, 152.4, 143.8, 136.2, 135.7, 134.5, 130.7, 129.2, 129.2, 129.1, 129.1, 128.9, 128.4, 117.2, 108.5, 62.3, 13.8. HRMS (ESI) calculated for [M+H]⁺ C₂₁H₁₆N₂O₂ m/z 329.1285, observed: 329.1288.

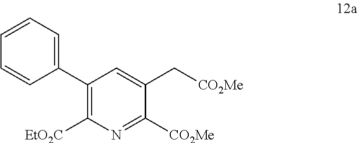 [SMILES: CCOC(=O)c1nc(C(=O)OC)c(CC(=O)OC)cc1-c1ccccc1]

Ethyl 6-Methylcarboxylato-5-(2-methoxy-2-oxoethyl)-3-phenylpyridine-2-carboxylate, 12a. Viscous liquid, (31.4 mg, 88% yield), 0.1 mmol scale reaction. Flash column chromatography conditions: hexane:ethyl acetate=5:1. ¹H NMR (500 MHZ, CDCl₃) δ 8.37 (s, 1H), 7.45-7.41(comp, 3H), 7.38-7.36 (comp, 2H), 4.36 (s, 2H), 4.18 (q, J=7.1, 2H), 3.91 (s, 3H), 3.72 (s, 3H), 1.04 (t, J=7.1, 3H). ¹³C NMR (126 MHz, CDCl₃) δ 171.0, 166.5, 165.8, 154.3, 151.3, 141.0, 137.0, 135.6, 128.7, 128.5, 128.4, 126.9, 62.0, 52.7, 52.2, 43.4, 13.7. HRMS (ESI) calculated for [M+H]⁺ C₁₉H₁₉NO₆ m/z 358.1285, observed: 358.1292.

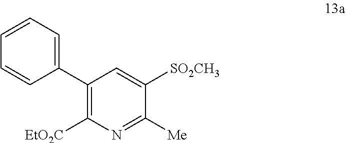 [SMILES: CCOC(=O)c1nc(C)c(S(C)(=O)=O)cc1-c1ccccc1]

Ethyl 6-Methyl-5-(methylsulfonyl)-3-phenylpicolinate, 13a. White solid, (27.1 mg, 85% yield), 0.1 mmol scale reaction. Flash column chromatography conditions: hexane:ethyl acetate=4:1. mp 102-104° C. ¹H NMR (500 MHZ, CDCl₃) δ 8.39 (s, 1H), 7.45-7.43 (comp, 3H), 7.39-7.36 (comp, 2H), 4.22 (q, J=7.1 Hz, 2H), 3.17 (s, 3H), 3.01 (s, 3H), 1.07 (t, J=7.1 Hz, 3H). ¹³C NMR (126 MHz, CDCl₃) δ 166.3, 156.0, 152.7, 139.5, 136.4, 136.2, 134.9, 128.9, 128.4, 62.3, 43.7, 23.4, 13.8. HRMS (ESI) calculated for [M+H]⁺ C₁₆H₁₇NO₄S m/z 320.0951, observed: 320.0959.

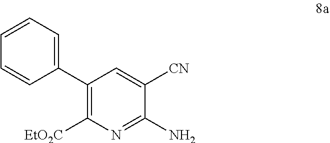 [SMILES: CCOC(=O)c1nc(N)c(C#N)cc1-c1ccccc1]

Ethyl 6-Amino-5-cyano-3-phenylpicolinate, 8a. White solid, (25.1 mg, 94% yield), 0.1 mmol scale reaction. Flash column chromatography conditions: hexane:ethyl acetate=4:1. mp 120-122° C. ¹H NMR (500 MHZ, CDCl₃) δ 7.81 (s, 1H), 7.44-7.40 (comp, 3H), 7.30-7.27 (comp, 2H), 5.57 (br s, 2H), 4.17 (q, J=7.2 Hz, 2H), 1.04 (t, J=7.2 Hz, 3H). ¹³C NMR (126 MHz, CDCl₃) δ 166.2, 157.7, 151.8, 143.7, 136.6, 128.8, 128.3, 128.2, 126.9, 115.8, 93.2, 62.2, 13.7. HRMS (ESI) calculated for [M+H]⁺ C₁₈H₁₃N₃O₂ m/z 268.1081, observed: 268.1080.

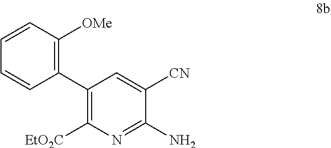 [SMILES: CCOC(=O)c1nc(N)c(C#N)cc1-c1ccccc1OC]

Ethyl 6-Amino-5-cyano-3-(2-methoxyphenyl)picolinate, 8b. White solid, (28.5 mg, 96% yield), 0.1 mmol scale reaction. Flash column chromatography conditions: hexane:ethyl acetate=4:1. ¹H NMR (500 MHZ, CDCl₃) δ 7.79 (s, 1H), 7.30 (t, J=7.9 Hz, 1H), 6.91 (dd, J=8.3, 2.6 Hz, 1H), 6.84 (d, J=7.6 Hz, 1H), 6.80-6.79 (m, 1H), 5.60 (br s, 2H), 4.17 (q, J=7.1 Hz, 2H), 3.81 (s, 3H), 1.05 (t, J=7.1 Hz, 3H). ¹³C NMR (126 MHz, CDCl₃) δ 166.2, 159.8, 157.7, 151.8, 143.7, 137.9, 129.8, 126.6, 120.7, 115.8, 113.9, 113.8, 93.1, 62.2, 55.4, 13.8. HRMS (ESI) calculated for [M+H]⁺ C₁₆H₁₅N₃O₃ m/z 298.1186, observed: 298.1185.

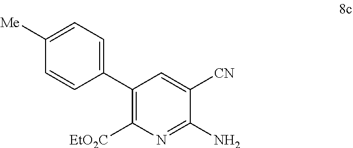 [SMILES: CCOC(=O)c1nc(N)c(C#N)cc1-c1ccc(C)cc1]

Ethyl 6-Amino-5-cyano-3-(p-tolyl)picolinate, 8c. White solid, (25.8 mg, 92% yield), 0.1 mmol scale reaction. Flash column chromatography conditions: hexane:ethyl acetate=4:1. ¹H NMR (500 MHZ, CDCl₃) δ 7.77 (s, 1H), 7.20 (d, J=7.8 Hz, 2H), 7.15 (d, J=7.8 Hz, 2H), 5.53 (br s, 2H), 4.17 (q, J=7.1, 2H), 2.38 (s, 3H), 1.07 (t, J=7.1, 3H). ¹³C NMR (126 MHz, CDCl₃) δ 166.3, 157.5, 151.7, 143.7, 138.1, 133.6, 129.4, 128.1, 126.9, 115.9, 93.2, 62.2, 21.3, 13.8. HRMS (ESI) calculated for [M+H]⁺ C₁₆H₁₅N₃O₂ m/z 282.1237, observed: 282.1236.

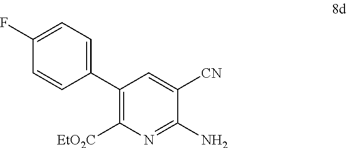 [SMILES: CCOC(=O)c1nc(N)c(C#N)cc1-c1ccc(F)cc1]

Ethyl 6-Amino-5-cyano-3-(4-fluorophenyl)picolinate, 8d. White solid, (23.9 mg, 84% yield), 0.1 mmol scale reaction. Flash column chromatography conditions: hexane:ethyl acetate=4:1. ¹H NMR (500 MHz, CDCl₃) δ 7.75 (s, 1H), 7.26-7.19 (comp, 2H), 7.11-7.08 (comp, 2H), 5.49 (br s, 2H), 4.17 (q, J=7.2 Hz, 2H), 1.07 (t, J=7.1 Hz, 3H). ¹³C NMR (126 MHz, CDCl₃) δ 166.0, 162.8 (d, J_{C-F}=250.0 Hz), 157.7, 151.8, 143.7, 132.7 (d, J_{C-F}=4.0 Hz), 130.1 (d, J_{C-F}=9.0 Hz), 125.8, 115.8 (d, J_{C-F}=22.0 Hz), 115.7, 93.3, 62.3, 13.8. HRMS (ESI) calculated for [M+H]⁺ C₁₅H₁₂FN₃O₂ m/z 286.0986, observed: 286.0985.

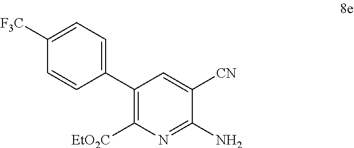 [SMILES: CCOC(=O)c1nc(N)c(C#N)cc1-c1ccc(C(F)(F)F)cc1]

Ethyl 6-Amino-5-cyano-3-(4-(trifluoromethyl)phenyl)picolinate, 8e. White solid, (27.2 mg, 81% yield), 0.1 mmol scale reaction. Flash column chromatography conditions: hexane:ethyl acetate=4:1. ¹H NMR (500 MHZ, CDCl₃) δ 7.78 (s, 1H), 7.67 (d, J=7.9 Hz, 2H), 7.39 (d, J=7.9 Hz, 2H), 5.74 (br s, 2H), 4.16 (q, J=7.2 Hz, 2H), 1.03 (t, J=7.2 Hz, 3H). ¹³C NMR (126 MHZ, CDCl₃) δ 165.6, 158.1, 151.4, 143.7, 140.5, 130.3 (d, J_{C-F}=32.0 Hz), 128.8, 125.7 (d, J_{C-F}=4.0 Hz), 125.6, 123.1 (d, J_{C-F}=270.0 Hz), 115.5, 93.4, 62.4, 13.7. HRMS (ESI) calculated for [M+H]⁺ C₁₅H₁₂F₃N₃O₂ m/z 336.0954, observed: 336.0949.

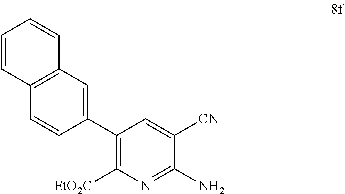 [SMILES: CCOC(=O)c1nc(N)c(C#N)cc1-c1ccc2ccccc2c1]

Ethyl 6-Amino-5-cyano-3-(naphthalen-2-yl)picolinate, 8f. White solid, (28.8 mg, 91% yield), 0.1 mmol scale reaction. Flash column chromatography conditions: hexane:ethyl acetate=3:1. ¹H NMR (500 MHZ, CDCl₃) δ 7.89-7.82 (comp, 4H), 7.74 (d, J=1.9 Hz, 1H), 7.53-7.51 (comp, 2H), 7.38 (dd, J=8.4, 1.9 Hz, 1H), 5.62 (br s, 2H), 4.13 (q, J=7.1 Hz, 2H), 0.94 (t, J=7.1 Hz, 3H). ¹³C NMR (126 MHZ, CDCl₃) δ 166.2, 157.7, 151.8, 144.0, 134.0, 133.3, 132.8, 128.5, 128.1, 127.8, 127.3, 126.8, 126.8, 126.7, 126.2, 115.8, 93.3, 62.2, 13.7. HRMS (ESI) calculated for [M+H]⁺ C₁₉H₁₅N₃O₂ m/z 318.1237, observed: 318.1232.

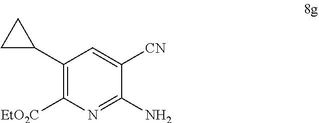 [SMILES: CCOC(=O)c1nc(N)c(C#N)cc1C1CC1]

Ethyl 6-Amino-5-cyano-3-cyclopropylpicolinate, 8g. White solid, (13.2 mg, 57% yield), 0.1 mmol scale reaction. Flash column chromatography conditions: hexane:ethyl acetate=3:1. ¹H NMR (500 MHZ, CDCl₃) δ 7.46 (s, 1H), 5.32 (br s, 2H), 4.44 (q, J=7.1 Hz, 2H), 2.13 (tt, J=8.4, 5.3 Hz, 1H), 1.41 (t, J=7.1 Hz, 3H), 0.96-0.90 (comp, 2H), 0.55 (dd, J=5.3, 1.4 Hz, 2H). ¹³C NMR (126 MHZ, CDCl₃) δ 165.9, 156.8, 153.1, 141.5, 127.7, 116.0, 93.4, 62.3, 14.3, 11.5, 7.4. HRMS (ESI) calculated for [M+H]⁺ C₁₂H₁₃N₃O₂ m/z 232.1081, observed: 232.1077.

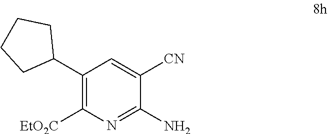 [SMILES: CCOC(=O)c1nc(N)c(C#N)cc1C1CCCC1]

Ethyl 6-Amino-5-cyano-3-cyclopentylpicolinate, 8h. White solid, (12.4 mg, 48% yield), 0.1 mmol scale reaction. Flash column chromatography conditions: hexane:ethyl acetate=4:1. ¹H NMR (500 MHZ, CDCl₃) δ 7.72 (s, 1H), 5.20 (br s, 2H), 4.42 (q, J=7.2 Hz, 2H), 3.20 (q, J=8.6 Hz, 1H), 2.06-2.00 (comp, 2H), 1.81-1.77 (comp, 2H), 1.69-1.62 (comp, 2H), 1.48-1.39 (comp, 5H). ¹³C NMR (126 MHz, CDCl₃) δ 166.2, 156.6, 151.9, 141.1, 130.2, 116.2, 93.8, 62.3, 53.6, 40.0, 34.6, 25.5, 14.3. HRMS (ESI) calculated for [M+H]⁺ C₁₄H₁₇N₃O₄ m/z 260.1394, observed: 260.1391.

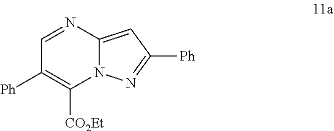 [SMILES: CCOC(=O)c1c(-c2ccccc2)cnc2cc(-c3ccccc3)nn12]

Ethyl 2,6-Diphenylpyrazolo[1,5-a]pyrimidine-7-carboxylate, 11a. White solid, (22.6 mg, 66% yield), 0.1 mmol scale reaction. Flash column chromatography conditions: hexane:ethyl acetate=4:1. mp 96-98° C. ¹H NMR (500 MHZ, CDCl₃) δ 8.58 (s, 1H), 8.02-8.00 (comp, 2H), 7.50-7.40 (comp, 8H), 7.09 (s, 1H), 4.45 (q, J=7.1 Hz, 2H), 1.24 (t, J=7.1 Hz, 3H). ¹³C NMR (126 MHZ, CDCl₃) δ 161.0, 157.2, 150.7, 149.1, 135.5, 133.3, 132.6, 129.3, 129.2, 128.9, 128.9, 126.9, 119.8, 94.2, 63.2, 14.0. HRMS (ESI) calculated for [M+H]^{+—}C₂₁H₁₇N₃O₂ m/z 344.1394, observed: 344.1389.

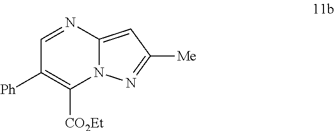 [SMILES: CCOC(=O)c1c(-c2ccccc2)cnc2cc(C)nn12]

Ethyl 2-Methyl-6-phenylpyrazolo[1,5-a]pyrimidine-7-carboxylate, 11b. White solid, (22.7 mg, 81% yield), 0.1 mmol scale reaction. Flash column chromatography conditions: hexane:ethyl acetate=3:1. ¹H NMR (500 MHz, CDCl₃) δ 8.52 (s, 1H), 7.47-7.44 (comp, 5H), 6.58 (s, 1H), 4.38 (q, J=7.1 Hz, 2H), 2.55 (s, 3H), 1.12 (t, J=7.1 Hz, 3H). ¹³C NMR (126 MHz, CDCl₃) δ 161.23, 156.50, 150.32, 148.74, 135.10, 133.51, 129.09, 128.95, 128.83, 119.16, 96.86, 63.16, 15.05, 13.83. HRMS (ESI) calculated for [M+H]⁺ C₁₆H₁₅N₃O₂ m/z 282.1237, observed: 282.1231.

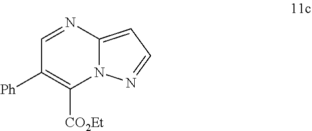 [SMILES: CCOC(=O)c1c(-c2ccccc2)cnc2ccnn12]

Ethyl 6-Phenylpyrazolo[1,5-a]pyrimidine-7-carboxylate, 11c. White solid, (20.8 mg, 78% yield), 0.1 mmol scale reaction. Flash column chromatography conditions: hexane:ethyl acetate=3:1. ¹H NMR (500 MHZ, CDCl₃) δ 8.61 (s, 1H), 8.20 (d, J=2.3 Hz, 1H), 7.49-7.45 (comp, 5H), 6.81 (d, J=2.3 Hz, 1H), 4.39 (q, J=7.1 Hz, 2H), 1.15 (t, J=7.1 Hz, 3H). ¹³C NMR (126 MHz, CDCl₃) δ 161.1, 150.8, 148.0, 145.8, 135.5, 133.2, 129.2, 129.0, 129.0, 120.1, 97.6, 63.3, 13.9. HRMS (ESI) calculated for [M+H]⁺ C₁₅H₁₃N₃O₂ m/z 268.1081, observed: 268.1075.

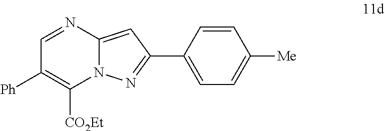 [SMILES: CCOC(=O)c1c(-c2ccccc2)cnc2cc(-c3ccc(C)cc3)nn12]

Ethyl 6-Phenyl-2-(p-tolyl)pyrazolo[1,5-a]pyrimidine-7-carboxylate, 11d. White solid, (25.0 mg, 70% yield), 0.1 mmol scale reaction. Flash column chromatography conditions: hexane:ethyl acetate=4:1. ¹H NMR (500 MHZ, CDCl₃) δ 8.58 (s, 1H), 7.98-7.87 (comp, 2H), 7.59-7.43 (comp, 5H), 7.35-7.24 (comp, 2H), 7.07 (s, 1H), 4.47 (q, J=7.2 Hz, 2H), 2.43 (s, 3H), 1.26 (t, J=7.2 Hz, 3H). ¹³C NMR (126 MHz, CDCl₃) δ 161.1, 157.3, 150.5, 149.1, 139.3, 135.4, 133.4, 129.8, 129.8, 129.5, 129.1, 128.9, 128.9, 126.9, 119.6, 93.9, 63.1, 21.5, 13.9. HRMS (ESI) calculated for [M+H]⁺ C₂₂H₁₉N₃O₂ m/z 358.1550, observed: 358.1541.

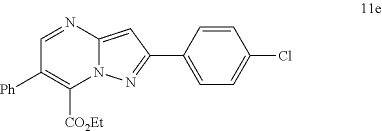 [SMILES: CCOC(=O)c1c(-c2ccccc2)cnc2cc(-c3ccc(Cl)cc3)nn12]

Ethyl 2-(4-Chlorophenyl)-6-phenylpyrazolo[1,5-a]pyrimidine-7-carboxylate, 11e. White solid, (24.1 mg, 64% yield), 0.1 mmol scale reaction. Flash column chromatography conditions: hexane:ethyl acetate=4:1. ¹H NMR (500 MHZ, CDCl₃) δ 8.58 (s, 1H), 7.94 (d, J=8.2 Hz, 2H), 7.49 (d, J=4.2 Hz, 5H), 7.43 (d, J=8.2 Hz, 2H), 7.05 (s, 1H), 4.44 (q, J=7.1 Hz, 2H), 1.23 (t, J=7.1 Hz, 3H). ¹³C NMR (126 MHz, CDCl₃) δ 161.0, 156.0, 150.9, 149.2, 135.3, 133.2, 131.1, 129.2, 129.1, 129.1, 128.9, 128.2, 128.1, 120.0, 94.2, 63.2, 13.9. HRMS (ESI) calculated for [M+H]⁺ C₂₁H₁₆ClN₃O₂ m/z 378.1004, observed: 378.1000.

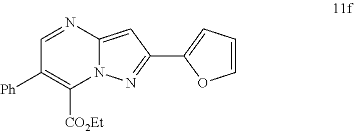 [SMILES: CCOC(=O)c1c(-c2ccccc2)cnc2cc(-c3ccco3)nn12]

Ethyl 2-(Furan-2-yl)-6-phenylpyrazolo[1,5-a]pyrimidine-7-carboxylate, 11f. White solid, (18.6 mg, 56% yield), 0.1 mmol scale reaction. Flash column chromatography conditions: hexane:ethyl acetate=4:1. ¹H NMR (500 MHZ, CDCl₃) δ 8.57 (s, 1H), 7.56 (dd, J=1.8, 0.8 Hz, 1H), 7.49-7.45 (comp, 5H), 7.00 (s, 1H), 6.97 (dd, J=3.4, 0.8 Hz, 1H), 6.53 (dd, J -3.4, 1.8 Hz, 1H), 4.42 (q, J=7.1 Hz, 2H), 1.18 (t, J=7.1 Hz, 3H). ¹³C NMR (126 MHz, CDCl₃) δ 160.9, 150.9, 149.4, 148.7, 148.1, 143.52, 135.4, 133.3, 129.3, 129.2, 129.0, 128.9, 128.9, 120.0, 111.9, 109.1, 93.9, 63.2, 13.8. HRMS (ESI) calculated for [M+H]⁺ C₁₉H₁₅N₃O₃ m/z 334.1186, observed: 334.1184.

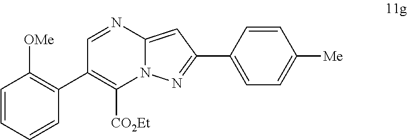 [SMILES: CCOC(=O)c1c(-c2ccccc2OC)cnc2cc(-c3ccc(C)cc3)nn12]

Ethyl 6-(2-Methoxyphenyl)-2-(p-tolyl)pyrazolo[1,5-a]pyrimidine-7-carboxylate; 11g. White solid, (26.7 mg, 69% yield), 0.1 mmol scale reaction. Flash column chromatography conditions: hexane:ethyl acetate=4:1. ¹H NMR (500 MHZ, CDCl₃) δ 8.56 (s, 1H), 7.93-7.87 (comp, 2H), 7.39 (t, J=7.9 Hz, 1H), 7.27 (s, 1H), 7.10-6.96 (comp, 4H), 4.46 (q, J=7.1 Hz, 2H), 3.86 (s, 3H), 2.41 (s, 3H), 1.27 (t, J=7.1 Hz, 3H). ¹³C NMR (126 MHz, CDCl₃) δ 161.09, 160.10, 157.35, 150.48, 149.14, 139.35, 135.45, 134.68, 130.26, 129.82, 129.56, 126.79, 121.27, 119.42, 114.55, 114.46, 93.89, 63.18, 55.51, 21.56, 13.99. HRMS (ESI) calculated for [M+H]⁺ C₂₃H₂₁N₃O₃ m/z 388.1656, observed: 388.1653.

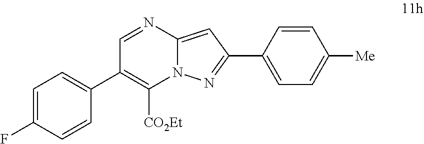 [SMILES: CCOC(=O)c1c(-c2ccc(F)cc2)cnc2cc(-c3ccc(C)cc3)nn12]

Ethyl 6-(4-Fluorophenyl)-2-(p-tolyl)pyrazolo[1,5-a]pyrimidine-7-carboxylate, 11h. White solid, (30.0 mg, 80% yield), 0.1 mmol scale reaction. Flash column chromatography conditions: hexane:ethyl acetate=4:1. ¹H NMR (500 MHZ, CDCl₃) δ 8.51 (s, 1H), 7.90 (d, J=8.0 Hz, 2H), 7.51-7.45 (comp, 2H), 7.27 (s, 1H), 7.19 (t, J=8.6 Hz, 2H), 7.05 (s, 1H), 4.45 (q, J=7.1 Hz, 2H), 2.41 (s, 3H), 1.27 (t, J=7.1 Hz, 3H). ¹³C NMR (126 MHz, CDCl₃) δ 163.3 (d, J_{C-F}=250.0 Hz), 161.0, 157.5, 150.3, 149.1, 139.4, 135.5, 130.8 (d, J_{C-F}=9.0 Hz), 129.7, 129.6, 129.4 (d, J_{C-F}=4.0 Hz), 126.8, 118.5, 116.3 (d, J_{C-F}=22.0 Hz), 94.0, 63.2, 21.5, 14.04. HRMS (ESI) calculated for [M+H]⁺ C₂₂H₁₈FN₃O₂ m/z 376.1456, observed: 376.1449.

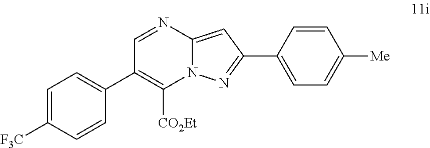 [SMILES: CCOC(=O)c1c(-c2ccc(C(F)(F)F)cc2)cnc2cc(-c3ccc(C)cc3)nn12]

Ethyl 2-(p-Tolyl)-6-(4-(trifluoromethyl)phenyl)pyrazolo[1,5-a]pyrimidine-7-carboxylate, 11i. White solid, (31.0 mg, 73% yield), 0.1 mmol scale reaction. Flash column chromatography conditions: hexane:ethyl acetate=4:1. ¹H NMR (500 MHZ, CDCl₃) δ 8.53 (s, 1H), 7.94 -7.86 (comp, 2H), 7.76 (d, J=8.1 Hz, 2H), 7.63 (d, J=8.0 Hz, 2H), 7.30-7.26 (comp, 2H), 7.07 (s, 1H), 4.47 (q, J=7.1 Hz, 2H), 2.41 (s, 3H), 1.27 (t, J=7.1 Hz, 3H). ¹³C NMR (126 MHz, CDCl₃) δ 160.7, 157.8, 149.8, 149.3, 139.6, 137.2, 135.7, 131.0 (q, J_{C-F}=44.0 Hz), 130.0, 129.6, 129.4, 126.8, 126.1 (q, J_{C-F}=4.0 Hz), 125.1 (q, J_{C-F}=270.0 Hz), 118.1, 94.2, 63.4, 21.6, 14.0. HRMS (ESI) calculated for [M+H]⁺ C₂₃H₁₈F₃N₃O₂ m/z 426.1424, observed: 426.1425.

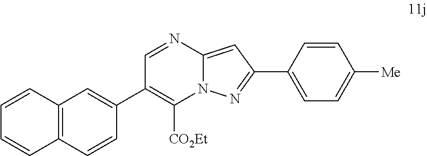 [SMILES: CCOC(=O)c1c(-c2ccc3ccccc3c2)cnc2cc(-c3ccc(C)cc3)nn12]

Ethyl 6-(Naphthalen-2-yl)-2-(p-tolyl)pyrazolo[1,5-a]pyrimidine-7-carboxylate; 11j. White solid, (29.3 mg, 72% yield), 0.1 mmol scale reaction. Flash column chromatography conditions: hexane:ethyl acetate=4:1. ¹H NMR (500 MHZ, CDCl₃) δ 8.66 (s, 1H), 8.00-7.95 (comp, 2H), 7.95-7.88 (comp, 4H), 7.60-7.55 (comp, 3H), 7.31-7.26 (comp, 2H), 7.07 (s, 1H), 4.43 (q, J=7.1 Hz, 2H), 2.42 (s, 3H), 1.20 (t, J=7.1 Hz, 3H). ¹³C NMR (126 MHZ, CDCl₃) δ 161.2, 157.4, 150.7, 149.1, 139.4, 135.6, 133.4, 133.1, 130.8, 129.8, 129.6, 129.1, 129.0, 128.5, 128.4, 128.3, 128.0, 127.9, 127.0, 127.0, 126.9, 126.9, 126.8, 126.7, 126.3, 126.2, 119.5, 94.0, 93.9, 63.2, 21.6, 14.0. HRMS (ESI) calculated for [M+H]⁺ C₂₆H₂₁N₃O₂ m/z 408.1707, observed: 408.1705.

Scale reaction (1.0 mmol). Triazine 1-oxide derivative 1a (1.0 mmol) was added all at once to a 10.0 mL dichloromethane solution containing ethyl 3-oxopentanoate (15.0 mmol) and a DBU (20.0 mmol). The reaction was continued for 10 minutes at room temperature. After full consumption of Triazine 1-oxide derivative 1a (monitored by TLC), solvent was evaporated under reduced pressure, and the crude product mixture was directly purified by flash chromatography (% ethyl acetate in hexanes=20-40%) to give the desired pyridine compound 4a (92%, 288.0 mg, 0.92 mmol).

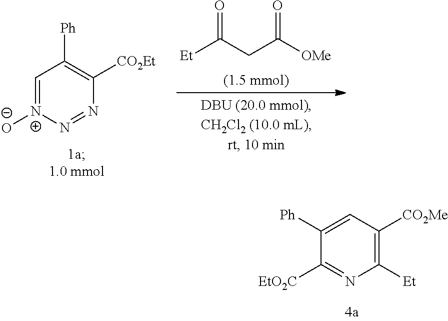 [SMILES: CCOC(=O)c1nn[n+]([O-])cc1-c1ccccc1 ; CCC(=O)CC(=O)OC ; CCOC(=O)c1nc(CC)c(C(=O)OC)cc1-c1ccccc1]

Crystallographic data. Single crystals of 3a were prepared by slow evaporation of a hexane/DCM solution. A suitable colorless needle-like crystal, with dimensions of 0.150 mm×0.061 mm×0.042 mm, was mounted in paratone oil onto a nylon loop. Single crystals of 4a were prepared by slow evaporation of a hexane/DCM solution. A suitable colorless block-like crystal, with dimensions of 0.245 mm×0.174 mm×0.116 mm, was mounted in paratone oil onto a nylon loop. Single crystals of 11i were prepared by slow evaporation of a hexane/DCM solution. A suitable colorless block-like crystal, with dimensions of 0.217 mm×0.074 mm×0.073 mm, was mounted in paratone oil onto a nylon loop. All data were collected at 100.0(1) K, using a XtaLAB Synergy/ Dualflex, HyPix fitted with CuKπ radiation ( λ=1.54184 Å). Data collection and unit cell refinement were performed using CrysAlisPro software. The total number of data were measured in the 6.46°<2θ<153.0°, 9.18°<2θ<153.1° and 8.57°<2θ<153.5° for compounds 3a, 4a and 11i respectively, using ω scans. Data processing and absorption correction, giving minimum and maximum transmission factors (0.784, 1.000 for compound (3a), 0.502, 1.000 for compound (4a) and 0.580, 1.000 for compound (11i)) were accomplished with CrysAlisPro and SCALE3 ABSPACK, respectively. The structure, using Olex2, was solved with the ShelXT structure solution program using direct methods and refined (on F²) with the ShelXL refinement package using full-matrix, least-squares techniques. All non-hydrogen atoms were refined with anisotropic displacement parameters. All hydrogen atom positions were determined by geometry and refined by a riding model. For compound 11i, the H-atoms on the methyl carbon, C23, observe a 50/50 positional disorder.

TABLE 5
Crystallographic data and structure refinement for Compounds 3a, 4a, 11i

| Compound number | 3a | 4a | 11i |
|---|---|---|---|
| Identification code | hpd526 | hpd599 | hpd611 |
| Empirical formula | C₁₉H₁₆N₂O₂ | C₁₈H₁₉NO₄ | C₂₃H₁₈F₃N₃O₂ |
| Formula weight | 304.34 | 313.34 | 425.40 |
| Crystal system | Monoclinic | Monoclinic | Monoclinic |
| Space group | P2₁/c | P2₁/c | P2₁/c |
| a (Å) | 13.8086(2) | 10.54414(10) | 9.35200(10) |
| b (Å) | 5.95817(7) | 15.67015(13) | 10.18750(10) |
| c (Å) | 18.7739(2) | 10.72943(10) | 20.6463(2) |
| α (°) | 90 | 90 | 90 |
| β (°) | 97.875(1) | 113.9853(12) | 92.3530(10) |
| γ (°) | 90 | 90 | 90 |
| Volume (Å³) | 1530.04(3) | 1619.72(3) | 1965.39 (3) |
| Z | 4 | 4 | 4 |
| ρ (calc.) | 1.321 | 1.285 | 1.438 |
| λ | 1.54184 | 1.54184 | 1.54184 |
| Temp. (K) | 100.0(1) | 100.0(1) | 100.0(1) |
| F(000) | 640 | 664 | 880 |
| μ (mm⁻¹) | 0.699 | 0.746 | 0.957 |
| Tₘᵢₙ, Tₘₐₓ | 0.784, 1.000 | 0.502, 1.000 | 0.580, 1.000 |
| 2θ_{range} (°) | 6.46 to 153.0 | 9.18 to 153.1 | 8.57 to 153.5 |
| Reflections collected | 14875 | 36026 | 36293 |
| Independent reflections | 3085 | 3348 | 4075 |
| | [R(int) = 0.0512] | [R(int) = 0.0393] | [R(int) = 0.0337] |
| Completeness | 95.8% | 98.1% | 98.4% |
| Data/restraints/parameters | 3085/0/210 | 3348/0/212 | 4075/1/300 |
| Observed data | 2762 | 3146 | 3845 |
| [I > 2σ(I)] | | | |
| wR(F² all data) | 0.0994 | 0.0883 | 0.0847 |
| R(F obsd data) | 0.0368 | 0.0338 | 0.0335 |
| Goodness-of-fit on F² | 1.03 | 1.05 | 1.04 |
| largest diff. peak and | 0.23/−0.19 | 0.30/−0.20 | 0.28/−0.30 |
| hole (e Å⁻³) | | | |

1. A chemical process comprising:
(a) combining a first 1,2,3-triazine 1-oxide reactant with a compatible second reactant in a solvent forming a reaction solution;
(b) incubating the reaction solution under conditions to form a product and nitrous oxide; and
(c) isolating the product.

2. The chemical process of claim 1, wherein the 1,2,3-triazine 1-oxide has the general structure of Formula I: 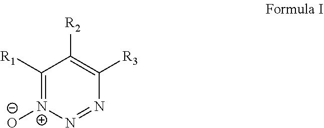
wherein R1, R2, and R3 are independently hydrogen, substituted or unsubstituted cycloalkyl, substituted or unsubstituted heterocyclyl, substituted or unsubstituted monocyclic aryl, substituted or unsubstituted polycyclic aryl, ester, or aldehyde.

3. The chemical process of claim 1, wherein the solvent is dioxane, tetrahydrofuran, methyl chloride, acetonitrile, or dichloromethane.

4. The chemical process of claim 1, wherein the reaction solution is incubated at a temperature of 10 to 35° C.

5. The chemical process of claim 1, wherein the reaction solution is incubated at a temperature of 20 to 25° C.

6. The chemical process of claim 1, wherein the product is a nitrogen containing monocyclic heterocycle or polycyclic heterocycle.

7. The chemical process of claim 1, wherein the product is isolated by chromatography.

8. The chemical process of claim 7, silica gel chromatography.

9. The chemical process of claim 1, wherein the reaction is substantially complete within 10 to 120 minutes.

10. The chemical process of claim 1. wherein the reaction is substantially complete within 20 to 40 minutes.
